(19) United States
(12) Patent Application Publication
(10) Pub. No.: US 2020/0115667 A1
(43) Pub. Date: Apr. 16, 2020

(54) VASCULARIZED MICROFLUIDIC PLATFORMS

(71) Applicant: BOARD OF REGENTS, THE UNIVERSITY OF TEXAS SYSTEM, Austin, TX (US)

(72) Inventors: Marissa Nichole RYLANDER, Austin, TX (US); Manasa GADDE, Santa Clara, CA (US); Rhys MICHNA, Austin, TX (US); Kameel ISAAC, Austin, TX (US); Alican OZKAN, Austin, TX (US); Sean BROCKLEHURST, Austin, TX (US); Neda GHOUSIFAM, Austin, TX (US)

(21) Appl. No.: 16/624,714

(22) PCT Filed: Jun. 21, 2018
(86) PCT No.: PCT/US2018/038734

§ 371 Date: Dec. 19, 2019

Related U.S. Application Data
(60) Provisional application No. 62/523,004, filed on Jun. 21, 2017.

(51) Int. Cl. C12M 3/06; B01L 3/00; C12M 3/00; C12M 1/00; C12M 1/12; C12N 5/09; C12N 5/071; G01N 33/50; B29C 33/38; B29C 33/40

(52) U.S. Cl. CPC C12M 23/16; B29L 2031/40; B01L 3/50273; C12M 21/08; C12M 29/00; C12M 25/14; C12N 5/0693; C12N 5/0691; G01N 33/5011; B29C 33/3842; B29C 33/40; B01L 2300/12; B01L 2200/12; B01L 2300/0832; B01L 2300/0809; C12N 2503/02; C12N 2533/52; C12N 2533/54; C12N 2533/56; C12N 2533/90; C12N 2513/00; B29K 2883/00; B01L 3/502707

(57) ABSTRACT

Provided herein are microfluidic vascularized platforms and methods of using the platforms. Further provided herein are skin model systems comprising hydrogel layers of cells.

The present application claims the priority benefit of U.S. Provisional Application Ser. No. 62/523,004, filed Jun. 21, 2017, the entire contents of which are hereby incorporated by reference.

The invention was made with government support under Grant No. R21 EB019646 awarded by the National Institutes of Health. The government has certain rights in the invention.

### BACKGROUND

### 1. Field

The present invention relates generally to the field of tissue engineering. More particularly, it concerns microfluidic techniques.

### 2. Description of Related Art

The continuous development of new fabrication techniques in the field of tissue engineering is leading to the creation of more advanced in vitro models to better understand the underlying influence of the microenvironment on human disease and tissue development. Three dimensional (3D) engineered tissue platforms have the ability to create physiologically representative features with an improved insight into the dynamic intricacies of the pathology of disease that is not possible with traditional two dimensional (2D) models. Some of the features not simulated in 2D culture include but are not limited to cell-cell and cell-matrix signaling, transport studies, and impact of mechanical and chemical gradients on cellular response. In vivo animal models offer more physiologically representative systems but limit control of microenvironmental conditions and the associated capability to determine their influence on physiological response dynamically and minimally invasively. Animal studies are costly for biological investigation and therapeutic refinement, limiting the ability to fully optimize therapeutics. Incorporation of microfluidic technology within 3D in vitro platforms allows long term cell culture and the ability to investigate the influence of flow and transport on dynamic cellular interactions in biological microenvironments. These types of 3D models promote cell growth and migration and are playing a growing role in the study of cancer biology due to their ability to examine the influence of individual factors on tumor progression.

Transport, cell-cell interactions, secretion of angiogenic growth factors, immune response, and other behaviors, that affect cell uptake of therapeutic drugs and play an intricate role in tissue pathology, are all variables of interest to be observed in a representative tumor environment.

Hyperpermeability of the vasculature within the tumor environment along with a lack of lymphatic drainage is responsible for elevated interstitial fluid pressure that can dramatically alter flow patterns as the tumor expands. These hydrodynamic behaviors may lead to increased expression of angiogenic factors and formation of microvessels inside the tumor allowing for tumor growth while transport and drug uptake can be reduced by the fluid dynamics of the tumor vasculature. Macromolecules and nanotherapeutics can fail to reach viable tumor cells due to the irregular extravasation and extravascular convection caused by the conditions of the tumor microenvironment. In order to study the influence of the vasculature on tumor development and transport of drugs, there has been a widespread expansion of in vitro platforms to incorporate channels to simulate vessels.

A number of microfluidic platforms have been developed to study the influence of the vasculature on normal and disease development. Zheng et al. have used additive tissue engineering techniques to develop 3D microfluidic vascular networks (μVN) in a collagen hydrogel for studying angiogenesis and thrombosis (Zheng et al., 2012).

In recent years, different methods have been proposed and discussed for the fabrication of microfluidic based vasculatures constructed within ECM. One method used frequently is to create a housing to encapsulate, polymerize, and pattern collagen to create vasculature using sterile needles (Tourovskai et al., 2014). Another platform was developed to mimic vascular tumor microenvironments that overcomes planar geometries inherent to conventional PDMS based devices to produce a more physiologically representative 3D cylindrical vascular microchannel (Szot et al., 2013). Although fluorinated ethylene propylene (FEP) tubing provided a robust infrastructure for maintaining vessel stability, the dimensions of the platform's tissue chamber could not be easily altered due to limitations set by using off the shelf FEP tubing to form the tissue chamber. The FEP also prevented the platform vasculature and tissue scaffolding from being scalable such that platform could not be built upon to form more advanced tissue systems for further studies. Introducing flow in the microchannel during live cell imaging on microscope stages presented challenges. The platform needed to be filled with water to match refractive indices in order to form an optically clear system for obtaining images. This requirement created the potential for leaks and limited resolution due to slight variations in refractive indices between the multiple mediums that composed the platform. Thus, there is an unmet need for enhanced microfluidic vascularized platforms.

### SUMMARY

In a first embodiment, there is provided a method of manufacturing a microfluidic device comprising obtaining a base mold with at least one protruding chamber and at least one rod which spans from one edge of the mold through the chamber to the opposite edge of the mold; casting a polymer solution onto the base mold; curing the polymer solution to form a solidified polymer mold; bonding the solidified polymer mold to a surface; inserting extracellular matrix hydrogel into the chamber; and removing the at least one rod once the extracellular matrix hydrogel has polymerized, thereby producing a microfluidic device comprising at least one chamber with at least one channel running through said chamber, wherein the at least one channel comprises an inlet port and an outlet port.

In some aspects, the base mold is an aluminum mold or polydimethylsiloxane (PDMS). In certain aspects, obtaining the base mold comprises performing micro-milling using a computer-numerical-control (CNC) machining system.

In some aspects, the chamber is cylindrical or rectangular. In certain aspects, the rod is a needle. In some aspects, the needle is a 20-30 gauge needle, such as a 21, 22, 23, 24, 25, 26, 27, 28, 29, or 30 gauge needle.

In certain aspects, the at least one channel has a diameter of 100 to 1000 μm, such as about 150, 200, 250, 300, 250, 300, 400, 500, 600, 700, 800, or 900 μm. The channel or additional channels in the device may have a diameter of 100-200, 200-300, 300-400, 400-500, 600-700, 700-800, or 900-1000 μm.

In further aspects, the base mold comprises 2, 3, 4, or 5 chambers, wherein each chamber has at least one rod running through said chamber. In some aspects, the chambers are in parallel. In some aspects, the chambers may be separated by the polymer, such as PDMS or a semi-impermeable membrane.

In some aspects, the at least one chamber has two (or 3 or 4) rods running through said chamber. In some aspects, the two rods have different diameters. In some aspects, the two rods have the same diameter.

In some aspects, the polymer solution comprises a silicon-based polymer. In certain aspects, the silicon-based polymer is polydimethylsiloxane (PDMS).

In certain aspects, curing comprises applying heat to the polymer solution. In some aspects, bonding comprises plasma treatment.

In some aspects, the surface is glass. In specific aspects, the glass is further defined a glass coverslip.

In additional aspects, the method further comprises treating the chamber with polyethleneimine (PEI) and/or glutaraldehyde before inserting the extracellular matrix hydrogel.

In some aspects, the extracellular matrix hydrogel comprises elastin, keratin, fibrin, fibronectin, laminin, hyaluronic acid, and/or collagen. In particular aspects, the extracellular matrix hydrogel comprises collagen, such as type I collagen. In some aspects, the extracellular matrix hydrogel comprises collagen and keratin, such as oxidized keratin (also referred to as keratose).

In some aspects, the extracellular matrix hydrogel further comprises one or more populations of cells. In some aspects, the one or more populations of cells are selected from the group consisting of tumor cells, hepatocytes, cardiomyocytes, keratinocytes, fibroblasts, endothelial cells, stem cells, and macrophages. Other cell type may be used depending on the desired model system.

In some aspects, the method further comprises injecting a population of cells into the channel. The rod or needle may be removed from the mold to inject the cells into the channel. The cells may be allowed to (e.g., under pre-conditioning flow) to form a layer, such as a monolayer, along the channel surface. Thus, the channel may be lined with cells to create a vascular structure or lymph vessels and or both. In some aspects, the population of cells comprises endothelial cells, such as TIME cells, HUVECs or lymphatic endothelial cells.

In some aspects, the method further comprises connecting the microfluidic device to a circulation system comprising one or more syringe pumps with controlled flow rates creating one or multiple channels. In some aspects, two or more channels are connected to flow in parallel or series. In some aspects, the flow rate for each of the channels is distinct creating pressure gradients. In some aspects, the flow rate for each of the channels is essentially identical. In other aspects, two or more channels are connected to flow in series. Flow rates can be designed to create fully functional, aligned, and endothelialized vessels.

In a further embodiment, there is provided a microfluidic device comprising a polydimethylsiloxane (PDMS) scaffold; a channel disposed within said PDMS scaffold; and at least one chamber in fluid communication with the channel or channels, wherein the chamber comprises an extracellular matrix hydrogel surrounding the channel.

In some aspects, the chamber is located in an interior region of said PDMS scaffold. In some aspects, the channel extends from the chamber to an external surface of said PDMS scaffold. In some aspects, the channel extends through said PDMS scaffold.

In certain aspects, the device is produced according to the methods of the embodiments.

In some aspects, the extracellular matrix hydrogel comprises elastin, fibrin, fibronectin, laminin, hyaluronic acid, keratin, and/or collagen. In some aspects, the extracellular matrix hydrogel comprises collagen. In some aspects, the collagen is type I collagen. In some aspects, the collagen is present in the hydrogel at a concentration of 5 to 15 mg/mL, such as about 6-7, 7-8, 8-10, 10-12, or 12-15 mg/mL. In some aspects, the collagen is present in the extracellular matrix hydrogel at a concentration of 6 to 12 mg/mL.

In some aspects, the device further comprises a population of cells dispersed within the extracellular matrix hydrogel of the chamber. In some aspects, the population of cells comprises tumor cells, cardiovascular cells, macrophages, kupfer cells, stellate cells, and/or hepatocytes. When modeling skin, keratinocytes, fibroblasts, and/or adipocytes can be incorporated in the extracellular matrix in a single or multi-layer structure.

In some aspects, the device comprise 2, 3, 4, or 5 chambers, wherein each chamber comprises a separate channel.

In some aspects, each chamber comprises a distinct population of cells within the extracellular matrix hydrogel. In some aspects, the at least one chamber comprises two channels. In some aspects, the two channels have distinct diameters. In some aspects, the two channels have essentially identical diameters.

In some aspects, the diameter of a channel is between 100 to 1,000 μm, such as 200 to 500 μm, such as about 150, 200, 250, 300, 250, 300, 400, 500, 600, 700, 800, or 900 μm. The channel or additional channels in the device may have a diameter of 100-200, 200-300, 300-400, 400-500, 600-700, 700-800, or 900-1000 μm.

In some aspects, the channel comprises a population of cells. In some aspects, the population of cells comprises endothelial cells or lymphatic endothelial cells. Immune cells or blood can be circulated through one or multiple channels.

In some aspects, the population of cells or populations of cells in the device comprises at least 1,000 cells, such as at least 5,000, 25,000, 50,000, 100,000, 200,000, 300,000, or 500,000 cells.

In some aspects, the device comprises one population of cells within the extracellular matrix hydrogel of the chamber and a second population of cells within the channel. In some aspects, the device comprises one population of cells within the extracellular matrix hydrogel of the chamber and a second population of cells within the channel. In some aspects, the device comprises tumor cells within the extracellular matrix hydrogel of the chamber and endothelial cells within the channel.

In some aspects, the cells within population comprise detectable markers. In some aspects, the first population of cells comprise a detectable marker distinct from the marker of the second population of cells.

In further embodiments, there are provided network platforms or branching vessels patterned after in vivo architecture. In one embodiment, there is provided a method of producing a network platform comprising filling a base mold, such as a well, with collagen and laying a flat PDMS piece on top to produce a flat collagen surface after polymerization. In some aspects, the well in the top component is aligned with a lithographically produced PDMS stamp that has a designed channel pattern. In some aspects, pins are inserted into the chamber to create an inlet and exit port before the chamber are filled with collagen and polymerized. In some aspects, after polymerization and removal of the PDMS and pins, the top and bottom components of the platform are stacked resulting in a network fully encased in collagen (Zheng et al., 2012; incorporated herein by reference). In some aspects, the process of forming biomaterials about a lithographic pattern produces a square cross-section in the channel. In some aspects, cells are seeded in the channel and a confluent endothelium is established to form a circular cross-section.

A further embodiment provides a method of evaluating a therapeutic or diagnostic agent comprising introducing the therapeutic or diagnostic agent to the flow of the microfluidic device of the embodiments and characterizing the effect of said therapeutic or diagnostic agent. In some aspects, evaluating comprises monitoring transport, uptake, toxicity, and/or efficacy of the therapeutic agent. In some aspects, characterizing is further defined as measuring cell viability, cell morphology, cell proliferation, and/or enzyme secretion.

In another embodiment, there is provided a method of measuring migration of a molecule (e.g., a cell, particle, bacteria, chemical, nanoparticle, or toxicant) comprising obtaining a microfluidic device of the embodiments, wherein the device comprises a chamber with at least two (or multiple) channels running through said chamber and a region of extracellular matrix hydrogel comprising a population of cells between said at least two (or multiple) channels; introducing media to the flow of the device; and monitoring the migration of a cell in the device. In some aspects, the channels comprise endothelial cells or lymphatic endothelial cells and the hydrogel comprises tumor cells and/or fibroblasts. In certain aspects, the hydrogel further comprises macrophages. In some aspects, the hydrogel comprises keratinocytes, fibroblasts, adipocytes, endothelial cells and/or tumor cells. In certain aspects, the media comprises cells, growth factors, cytokines, hormones, antibodies, drugs, and/or enzymes. In some aspects, the media comprises or consists of whole blood.

In a further embodiment, there is provided a multi-layer hydrogel system for modeling skin comprising a first layer of collagen hydrogel comprising keratinocytes (e.g., to mimic the epidermis), a second layer of collagen hydrogel comprising fibroblasts and/or endothelial cells (e.g., cultured in the collagen or single or multiple endothelialized channels forming the dermis), and a third layer of collagen hydrogel comprising adipocytes, endothelial cells, an endothelialized blood vessel, or lymph channels (e.g., to mimic the subcutaneous layer). In some aspects, the first, second, and/or third hydrogel layer further comprises keratin, melanocytes, hair follicles, and/or neural cells. In some aspects, the percentage of collagen and/or additional ECM components, such as keratin, may be varied within the different layers. In some embodiments, the system may be used as a representative skin model. This skin model can be used to assess injury from chemical or thermal insults or transport of chemicals, bacteria, cells, or toxicants across the skin. Assessment of burn or blast injury is also a suitable use of the technology. In some aspects, the extracellular matrix hydrogel comprises collagen and keratin, such as oxidized keratin (also referred to as keratose).

In further embodiments, there is provided a provided an extracellular matrix comprising collagen and keratose. In some aspects, the extracellular matrix is used in a model provided in the present embodiments and aspects thereof.

As used herein, “essentially free,” in terms of a specified component, is used herein to mean that none of the specified component has been purposefully formulated into a composition and/or is present only as a contaminant or in trace amounts. The total amount of the specified component resulting from any unintended contamination of a composition is therefore well below 0.05%, preferably below 0.01%. Most preferred is a composition in which no amount of the specified component can be detected with standard analytical methods.

As used herein the specification, “a” or “an” may mean one or more. As used herein in the claim(s), when used in conjunction with the word “comprising,” the words “a” or “an” may mean one or more than one.

The use of the term “or” in the claims is used to mean “and/or” unless explicitly indicated to refer to alternatives only or the alternatives are mutually exclusive, although the disclosure supports a definition that refers to only alternatives and “and/or.” As used herein “another” may mean at least a second or more.

Throughout this application, the term “about” is used to indicate that a value includes the inherent variation of error for the device, the method being employed to determine the value, or the variation that exists among the study subjects.

Other objects, features and advantages of the present invention will become apparent from the following detailed description. It should be understood, however, that the detailed description and the specific examples, while indicating preferred embodiments of the invention, are given by way of illustration only, since various changes and modifications within the spirit and scope of the invention will become apparent to those skilled in the art from this detailed description.

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein.

FIGS. 1A-1G: Design of platform and fabrication of single vessel microfluidic collagen platform and perfusion setup. (a) CAD design of PDMS mold and PDMS tissue chamber with 22G inlets. (b) Machined aluminum mold, PDMS chamber produced from aluminum mold and glass cover slip. (c) Tissue chamber filled with collagen around a 22G needle (d) Collagen hydrogel with channel after polymerization and removal of needle. (e) Setup of syringe pump flow system with bubble traps for perfusion through the hydrogel. (f) Close-up view of platform with 0.5″ 22G needles inserted into the chamber inlet and outlet for preconditioning. (g) CAD design of PDMS tissue chamber and schematic of cells within vessel.

FIGS. 2A-2G: Design of network platform and fabrication of microvascular network collagen hydrogels and perfusion setup. (a) CAD design of platform components and assembly. (b) Machined aluminum and polycarbonate components, PDMS stamps produced using lithographic techniques, and off the shelf parts. (c) Tissue chambers filled with collagen. (d) Collagen hydrogel with channel after polymerization and stacking of layers. (e) Setup of syringe pump flow system with bubble traps for perfusion through the network. (f) Close-up view of threaded inserts for attaching flow systems to the platform. (g) Gravity driven reservoir flow system.

FIGS. 3A-3I: Showcase of single vessel platform design improvements. (a) Original Rylander lab single vessel platform. (b) New scalable mold for making single vessel platforms. (c) CAD model of proposed multi-chamber vascularized tissue model. (d-f) Confocal single plane image of endothelialized vessels walls at the widest diameter formed using a 22, 25, and 30 gauge needle respectively. (g) Concept mold and platform for making dual channel platform. (h) Confocal single plane image of endothelialized 22 gauge dual channel tumor model. (i) Schematic of fabrication of single vessel platform.

FIGS. 4A-4F: Imaging of cell viability and confluency within the single vessel platform obtained upon immediate completion of preconditioning protocol and staining. Scale bars are 500 μm. (a) Front view of co-culture viability test. (b) Top view of f-actin stained mono-culture endothelium. (c) Isometric 3D view of endothelium surrounded by cancer cells. (d) Top view of f-actin stained endothelium in a co-culture environment. (e,f) Cross sectional view of SEM images of the endothelium showing a cylindrical endothelium in the TIME monoculture (e) and MDA-MB-231 and TIME cell co-culture (f).

FIG. 5: Images of 70 kDA fluorescent dextran diffusing through a single vessel platform over 2 hours at a flow rate of 260 μl/min. Permeability coefficients after 2 hours of flow plotted for acellular, endothelium, and co-culture endothelium with cancer cells in the hydrogel (n=3, p<0.05).

FIGS. 6A-6H: In vitro tumor microenvironment microvascular network. (a) Isometric view of network co-culture of TIME cells and MDA-MB-231 cells. (b) Top view of the co-culture network. (c) Cross section view of channel near inlet. (d) Cross section view of 4 channels. Scale bar is 100 μm. (e) Transport of blue microspheres through the network. (0 Model predicted velocity magnitude inside tumor microenvironment microvascular network using finite element method (FEM) simulations. Scale bar is 500 μm. (g, h) SEM images the microchannels in the network platform showing a patent and continuous endothelium at the corners (g) and in one of the channels (h). Scale bar is 20 μm.

FIGS. 7A-7E: In vitro recreation of an in vivo tumor. (a) two-photon image showing human colon carcinoma vasculature in mice at day 0 (Tong et al., 2004). Circled region indicates areas difficult to recreate in vitro. (b) Engineered microfluidic tumor microenvironment capturing the geometry of an in vivo tumor. Scale bar is 100 μm. (c) Obtained velocity magnitude inside in vivo tumor microenvironment microvascular using FEM simulations. Scale bar is 500 μm. (d) Normalized intensity prolife of 70 kda dextran particles through in vivo vascular patterned platform as a function of time, scale bar is 400 μm. (e) (i) Transport of blue particles through the in vivo vascular patterned platform consisting of a co-culture of MDA-MB-231 cells and TIME cells, scale bar is 250 μm. (ii, iii) Close up images of two different areas in the platform revealing aggregation of the particles, scale bars are 100 μm.

FIG. 8: Development of the endothelium throughout the flow protocol in a TIME cell only.

FIGS. 9A-9D: In vitro vascularized breast tumor platforms consisting of monoculture of TIME cell seeded lumen (a) or co-culture of GFP labeled MDA-IBC3 (b), SUM149 (c), MDA-MB-231(d) tumor cells around a TIME cells seeded lumen; scale bar: 500 μM.

FIGS. 10A-10C: Immunofluorescent staining of the endothelium: (a) PECAM-1 staining of endothelial cell-cell junctions with DAPI staining of cell nuclei; scale bar: 100 μm. (b) F-actin and DAPI staining; scale bar: 200 μm. (c) SEM images of endothelial morphology and adhesion; scale bar: 10 μm.

FIG. 11: Endothelium coverage of the vessel lumen for different co-culture platforms; *p<0.05, ** p<0.01.

FIG. 12: Measured effective permeability coefficient for different co-culture platforms; *p<0.05.

FIG. 13: VEGF expression at 72 and 78 hour time points; statistical significance at 72 hours is represented by solid lines while dotted lines represent 78 hours. *p<0.05, **p<0.01.

FIGS. 14A-B: (a) SEM images of tumor cells and collagen matrix. (b) Collagen matrix porosity measurements, *p<0.05, **p<0.01

FIG. 15: Fabrication steps of vascularized tissue microenvironment. PDMS was mixed with curing agent and poured into aluminum mold shown in (I) and baked. Inlet and outlets were patterned around a 22G or 27G needle and housing was patterned around aluminum extrusion shown in (II). PDMS was peeled off from aluminum mold and bonded to glass slide and platform shown in (III) was treated with PEI, glutaraldehyde and DI H₂O. Same platform cavity was filled with collagen mixture with requested cell line. To form channel to simulate the vessel, the needle was inserted (IV). Needle size were selected depending on desired wall shear stress. Needle was removed after polymerization of collagen (V) and preconditioned after injection of endothelium cells for 72 hours to form a vessel (VI).

FIGS. 16A-16D: Design and fabrication of multi-chamber microfluidic platform and perfusion setup. (a) CAD design of aluminum mold with 22G inlets. (b) Schematic of liver-breast tumor microenvironment interaction and transport. (c) Close-up view of platform with 0.5″ 22G pins inserted into the chamber inlet and outlet for flow preconditioning and particle testing. Confocal images show preconditioned liver and breast vessels with GFP tagged cancer cells and FITC tagged Anti-Albumin immunostained healthy liver cells. Scale bar is 500 (d) Shear stress profile across tumor and healthy vessels obtained using finite element method simulations. Targeted physiological wall shear stresses for tumor and liver are 1 and 4 dyn/cm², respectively, consequently, vessel diameters for healthy liver and tumor are 435 and 711 μm.

FIG. 17: Morphology of MDA-MB-231, THLE-3, and C3Asub28 cell lines within the avascular microenvironments. F-Actin and DAPI stained samples shows aggregation over time. Scale is 20 μm. SEM images shows outline of a single cell in each matrices by day 3. Scale is 10 μm.

FIG. 18: Cell growth over time within the avascular platforms with initial seeding density of 1×10⁶ cells/ml for MDA-MB-231, THLE-3, and C3Asub28 cell lines within the tissue microenvironments over 3 days. Cell concentration was normalized to Day 0. Data shown are mean SD (* p<0.05).

FIGS. 19A-19B: Albumin expression and release from healthy liver cells within THLE-3/TIME vascularized microenvironments. (a) FITC tagged antialbumin immunostained healthy liver cells overlaid with bright field image. Scale is 10 μm (b) Albumin release from THLE-3/TIME vascularized microenvironment during preconditioning period and physiological wall shear stress. Data shown are mean_SD. (*** p<0.005, **** p<0.001).

FIG. 20: Confocal images of red labeled endothelial cells in each vascularized in vitro microenvironments. Control − refers to TIME monoculture and Control + refers to TIME monoculture treated with TNFα. Vasculature diameter varies between 411-450 and 700-750 μm for THLE-3 and all other cell lines, respectively. Scale bar is 500 μm.

FIGS. 21A-21D: Permeability and porosity for different cell culture microenvironments. (a) Vessel porosity quantified using confocal microscopy images. (b) Fiber structure of different microenvironments obtained with SEM. (c) Quantified ECM porosity of each microenvironment using SEM images. (d) Permeability of endothelial lumen for different particle sizes and vascularized microenvironments. Control + refers to TNFα treated vasculature and Control − refers to TIME monoculture. Data shown are mean SD. *p<0.05, **p<0.01, *** p<0.005, **** p<0.001, n.s.: not significant.

FIG. 22: Diffusion curves of fluorescent dextran particles (3 and 70 kDa) with respect to time and position across ECM boundaries for Control −, healthy liver (THLE-3), liver (C3Asub28), and breast (MDA-MB-231) tumor microenvironments. Fluorescence intensity profiles of 3 experiments were averaged. Dashed lines represent vessel boundaries.

FIG. 23: Diffusion curves of fluorescent dextran particles (3 and 70 kDa) with respect to time and position across ECM boundaries for secondary compartments of breast (MDA-MB-231) and liver (C3Asub28) carcinoma and healthy liver (THLE-3) microenvironments. Fluorescence intensity profiles of 3 experiments were averaged. Dashed lines represents vessel boundaries. The nanoparticle circulation order is presented as in the figure.

FIG. 24: Diffusion rate in vessel and ECM in response to different sequence of perfusion in the tissue microenvironments. First microenvironment accumulation in the (a) ECM and (b) vessel for MDA-MB-231, C3Asub28, THLE-3 microenvironments and negative control. Second microenvironment accumulation in the (c) ECM and (d) vessel for MDAMB-231, C3Asub28, and THLE-3 microenvironment interactions when particles are introduced to either the liver or tumor first. Microenvironment order is presented as in the figure. Data shown are mean_SD. *: p<0.05, **: p<0.01, ***: p<0.005, ****: p<0.001, n.s.: not significant.

FIGS. 25A-25F: Multi-organ platform: (a) plug-and-play connection in a circulation system, (b) close-up view of vascularized platforms with varying channel diameters, and (c-e) exploded view of system. (f) schematic of platform with four compartments to make multiple platform molds at once. 2 platforms will be placed on top of each other with a semipermeable membrane between the layers.

FIG. 26: Immunofluorescence confocal microscopy image of a full multilayer skin tissue model after 7 days of growth. Side view. Keratinocytes (top), fibroblasts (middle), and vascular endothelial cells (bottom) are shown. The distinct dermis and epidermis layers can be clearly seen as cells remained in their appropriate layers. The dermis layer was made thinner than usual to improve the quality of imaging. The endothelial cells formed a continuous endothelium on the bottom of the dermis layer, representing the combined surface area of microvasculature present within the dermis in vivo.

FIGS. 27A-27B: (a) Transwell multi-layer system schematic. (b) Multi-layer platform with endothelial cells, fibroblasts, in the dermal layer, and keratinocytes in the epidermal layer.

FIGS. 28A-28B: (a) Schematic of multi-layer comprehensive tumor/skin vascularized platform. (b) Confocal image of the combined dermal and tumor platform with vessels.

FIGS. 29A-29C: (a) Single-layer vasculature for skin with staining of cytokeratin 14 for basal, undifferentiated keritanocytes. (b) Vasculature model stained for caspase 14 for differentiated keratinocytes. (c) Multi-layered vascularized platform with all 3 cell types: keratinocytes, fibroblasts and endothelial cells.

FIG. 30: Dual channel platform with vascular (right) and lymphatic (left) endothelial channels surrounded by a collagen matrix seeded with IBC cells.

FIGS. 31A-31C: Dual channel platform with mammosphere formation: (a) Center plane of the dual lymphatic (left) vascular platform (right) showing tumor emboli (denoted by white arrows) invading into the channels, scale bar is 500 μm. (b) Migration of cancer cells in response to a growth factor gradient, scale bar is 50 μm. (c) Confocal image of skin platform.

FIGS. 32A-32C: SEM images of 100% collagen (a), 50/50 collagen/keratin (b), and 20/80 collagen keratin (Cc at different magnifications, ranging from highest to lowest from left to right.

FIGS. 33A-33E: SEM images of 100% collagen (a), 50/50 collagen/keratin (b), and 20/80 collagen/keratin gels (c) compared with the same image when threshold was applied in ImageJ® to determine percent porosity (d). Fiber width of 100% collagen, 50/50 collagen/keratin, 20/80 collagen/keratin gels (e).

FIG. 34: Protein denaturation temperature of hydrogels. Single-factor ANOVA was performed between each group. *=p<0.01.

FIGS. 35A-35B: (a) Cell viability of NHDFs in 1.5 and 3 mg/ml gels and (b) MDA-MB-321 breast cancer cells in 5 and 7 mg/ml gels. Single-factor ANOVA was performed with *=p<0.01 and #=p<0.05.

FIG. 36: Phalloidin-488 stained fibroblasts in various hydrogels imaged using a confocal microscope.

FIG. 37: Cytokeratin (undifferentiation marker), caspase 14 (differentiation marker) and involucrin (differentation marker) was expressed in day 7 post-air exposure of keratinocytes seeded on 100% and 50/50 C/K hydrogels. Although caspase 14 was expressed in both types of gels, involucrin, a marker of late keratinocyte differentiation, was expressed more in 50/50 C/K gels, indicating that the presence of keratin induces further differentiation of keratinocytes.

FIGS. 38A-38B: The co-culture microfluidic model was utilized to quantify SWNH-QD extravasation from the central microvessel and to determine if the effect of mild hyperthermia on this mass transport process. To accomplish this, a permeability coefficient (Pd) was calculated in a region of interest in the platform (a), The ROI was set so endothelialized microvessel was located in the center of the region. The environmental temperature was maintained with an incubated stage during the 1 h exposure and monitored using multiple thermocouples throughout the study, ensuring steady-state temperature. Images were taken every 5 minutes to calculate Pd, and the temperature dependence of SWNH-QDs was successfully evaluated at 37° C. and 42° C. The results from this study indicates that the permeability of SWNH-QDs significantly increases in the 3D cylindrical co-culture tumor model when exposed to mild hyperthermia (42° C.) to 44.62±8.83 μm*10²/s, as compared to an average Pd of 29.14±5.2 μm*10²/s at for the model at normothermia (37° C.) for 1 h (b) p <0.05, Students T-test.

FIG. 39: The 3D platform was utilized to understand how thermally enhanced nanoparticle vascular-permeability arises. In particular, it was sought to understand the endothelial response to 42° C., as the confluent monolayer of endothelial cells acts as the primary barrier to diffusion into the interstitial collagen matrix. Using F-actin and DAPI stain, it was sought to determine if microscopic changes such as cytoskeletal dysregulation could leads to reduce in cell-cell interactions or the complete obliteration of the endothelium. At 42° C. no discernible loss was observed in endothelial cell density, determined by the presence of evenly distributed cell nuclei throughout the vessel wall and no loss in cell density. However, a marked disruption of the endothelial cytoskeleton was observed for platforms exposed to 42° C., shown in the F-Actin staining.

FIGS. 40A-40C: (a) Radial profile from a high resolution image taken during SWNH-QD perfusion into the co-culture platform. (b) No significant changes in relative intensity values are seen in the tumor monoculture at the two temperatures. (c) Representative images after staining for F-actin in samples exposed to 42° C. for 1 h.

### DESCRIPTION OF ILLUSTRATIVE EMBODIMENTS

Certain embodiments of the present disclosure provide microfluidic devices, methods of fabricating the devices, as well as methods of using the microfluidic devices. Accordingly, some embodiments provide a physiologically representative three-dimensional in vitro microfluidic vascularized platform of varying complexity that is simple to fabricate and enables high throughput investigation of biological processes and optimization of diagnostics and therapeutics. The in vitro vascularized platforms may consist of single or multiple confluent cylindrical endothelium blood or lymph vessels surrounded by a collagen, keratin, or collagen/keratin blend extracellular matrix seeded with tissue specific cells recreating the tissue-blood vessel interface.

Accordingly, in some embodiments, there is provided herein a method and 3D physiologically representative, high throughput platform for representing vascularized tissues and tumors. The present platform can have the capability to eliminate artificial boundaries, provide a physiologically representative extracellular matrix to facilitate realistic cell growth and response, create a functional endothelium for study of transport and cell migration, and replicate complex vessel and tissue architecture inexpensively. By changing out cells, different tissues can be replicated and the platform can be used for diagnostic, therapeutic, and device development.

Thus, further embodiments provide methods for cardiovascular applications for stent and drug design. The present platform can also be used for cancer drug discovery and optimization is possible by allowing the influence of therapeutic/diagnostic properties on drug localization, specificity, and efficacy, dosing, and time of delivery to be studied. By creating vascularized tumor, liver, and heart in series or parallel toxicity to heart and metabolism by the liver as a function of therapeutic properties and dose can be optimized. Multiple vascularized platforms are provided herein to model the tumor, and distant metastatic sites (bone, brain etc) to enable identification of new molecular diagnostic and therapeutic targets for aggressive, metastatic disease. Also by creating dual vessels in the platform the influence of biochemical/pressure gradients on drug delivery, efficacy, tumor metastasis can be studied in the context of different therapeutics. Multiple layer vascularized skin platforms are also provided, such as for optimizing protective garments to prevent burn injury. In addition, patient specific vascularized tissues can be created for personalized diagnostics and therapeutics

In some aspects, the present platforms are produced by subtractive tissue engineering fabrication methods (e.g., micro-milling techniques) and lithographic techniques with additive tissue engineering methods. The present platform can overcomes restriction to the geometry and size of the tissue chamber while still creating a viable in vitro tumor microenvironment that can easily interface with imaging setups to complete studies of the microenvironment. The bonding of a PDMS chamber directly to a glass cover slip allows the hydrogel to be positioned closer to microscope objectives and eliminates the necessity of using water to create an optically clear system for imaging. The platforms can be customized to represent different tissue types and the complexity of the platform can be varied from single vascularized vessel systems to more intricate vascularized microfluidic networks that capture microvasculature representative of patient tissues for the first time. Since the tissue platform itself may be entirely made of collagen extracellular matrix, cells contained in the system maintain all their biological functions (e.g., growth, migration, gene expression) enabling representative biological and pathological processes to be studied and the response to diagnostics and therapeutics to be optimized in a realistic manner.

In some aspects, the present platforms are functional and physiologically representative endothelium that are created within the vascular channels. In some embodiments, the presence of a functional vasculature in connection with a tissue enables therapeutics and diagnostics to be optimized by honing the properties of the agent (e.g., size, surface properties, shape) to maximize transport/localization in turn diagnostic sensitivity and therapeutic efficacy. The design of this system when coupled with labeled cells can enable dynamic imaging of transport of the diagnostic/therapeutic and real time response (e.g., apoptosis, migration, and necrosis) of the cells targeted enabling optimization of therapeutic features to maximize toxicity. In addition to properties of the therapeutic being optimized, the ideal timing of the drug can be determined based on dynamic understanding of pathological state. Thus, the present system offers a high throughput and inexpensive alternative to animal testing enabling each aspect of therapeutic and diagnostic agent to be optimized.

In one embodiment, there are provided complex vascularized in vitro models to mimic the tumor microenvironment which can be tailored to represent various aggressive breast tumors such as inflammatory breast cancer. Features of these platforms include a continuous, aligned endothelium that allows for cell-cell interactions between vasculature and tumor cells. It was demonstrated the phenotype of the cancer has an influence on the leakiness of the endothelium, initiation of angiogenesis and modulation of the surrounding tumor microenvironment.

In some embodiments, a platform is provided for fabrication of a single endothelialized microchannel encased within a collagen platform hosting tumor cells, such as breast cancer cells. The microfluidic device was developed and utilized to study the influence of cellular interaction on transport phenomenon through vasculature in a hyperpermeable tumor microenvironment. This platform relies on subtractive tissue engineering fabrication techniques. Through confocal imaging, it was demonstrated that the platform produces enhanced leakiness recapitulating physiological features of the tumor microenvironment. The influence of tumor endothelial interactions on transport of particles was also demonstrated.

In certain embodiments, the housing material mold for the platform is fabricated using micro-milling techniques. This method may decrease fabrication time and eliminate the requirement of clean room facilities, multistep fabrication processes, and expensive reagents to produce a vascularized microchannel. The platform can be easily customized to alter geometries of the tissue chamber and vessel size while maintaining a continuous lumen. Additional benefits include reduction in the amount of collagen and reagents required to fabricate the platform. The platform can also establish continuous live cell imaging of particle transport.

Additional embodiments concern fabrication of platforms with vasculature recreated from patient specific data that can be used for developing personalized treatments. The platforms may possess a functional and aligned endothelium without the presence of an artificial boundary as the cells are cultured directly on collagen. This can enable realistic transport of therapeutics and diagnostics to be studied within a tissue and the transport/targeting properties of these agents to be optimized in an inexpensive, and high throughput manner.

Further embodiments enable use of the microfluidic devices provided herein for the identification of new diagnostic/therapeutic targets, optimization of diagnostic/therapeutics agents, and realization of the ultimate goal of personalized treatment plans. In some embodiments, applications include creation of the blood brain barrier for use in homing drug transport and localization.

In further embodiments, multiple vascularized tissues are connected to enable the influence of a diagnostic/therapeutic on the target tissue and toxicity to other collateral tissues/organs (e.g., heart, liver) to be studied dynamically enabling optimization of agent properties and dosing regimens. In one particular embodiment, the device comprises vascularized tumor, liver, and heart that enables study of the transport and therapeutic efficacy and toxicity to the liver and heart.

In some embodiments, the devices provided herein may be used to study the influence that metabolism by the liver has on tissue response and drug effectiveness. This holds particular promise for chemotherapeutic optimization where the goal is to minimize collateral toxicity to the heart and liver while maximizing therapeutic efficacy to the tumor of interest. Another embodiment provides devices with multiple vascularized compartments including the tumor of interest and potential metastatic sites of cancer (e.g., bone, brain, lung) to enable homing of the tumor and diagnostic and therapeutic optimization to effectively diagnose and treat varying stages of disease and identify new molecular markers that signify poor prognosis.

The vascularized platforms can also be adapted to model the interactions between tumors, blood vessels, and lymph vessels which has tremendous promise for tumors such as aggressive tumors that metastasize through the lymph system. In particular embodiments, the system provided herein is capable of replicating complex vascular networks and tissues particularly patterned after patient imaging data (e.g. CT and MRI) to enable personalized treatment plans. The platforms may also be used for cardiovascular applications in which the presence of a vessel and surrounding heart need to be modeled to optimize stent design or placement or drug delivery.

Further, major challenges also arise due to delivery drugs based on adverse pressure and biochemical gradients within a tissue or tumor. Thus, in some embodiments, the present devices may be used as multi-vessel tissue platforms enabling optimization of diagnostic and therapeutics.

Additionally, in some embodiments, there is provided a second platform capable of combining lithographic techniques with additive tissue engineering methods to create intricate endothelialized microfluidic networks that capture the more complex geometries of tumor microvasculature representative of patient tumors. By modeling microvascular networks after in vivo tumors, patient specific in vitro platforms may be created that can be used to develop personalized patient treatments.

In some embodiments, the device comprises a single vessel or multiple vessels, such as dual vessels to study migration of cells. In some embodiments, the collagen used in the present devices is obtained from an animal. For example, the collagen may be obtained from rats, particularly rat tails.

In further embodiments, there are provided multi-layer vascularized skin platform containing blood vessels and lymph vessels, such as to study burn injury characteristics including increased capillary permeability, leakage between dermis and epidermis, and destroyed tissue becomes eschar. The healing of burn wounds comprise an inflammatory phase (e.g., chemotactic factors attract immune cells), proliferative phase (e.g., cell proliferation, tissue regrowth), and a remodeling phase (e.g., fibrous structural proteins and scar tissue formation). The skin platform may be evaluated using live/dead staining, GFP expression, heat shock proteins, such as 27, 47, 60, and 70, CD31 staining, and cell proliferation. Contact burn testing may comprise burning the sample by contact with a heated copper rod. The temperature and exposure time may be controlled. The damage to gels may be visible through live/dead staining and imaging. The gels can remain viable for at least 3 days after burning. The skin platform technology provided herein possesses a functioning vasculature unlike existing skin systems. It enables the dynamic study of skin infection as a function of barrier function, vasculature perfusion, and immune response which will yield key insights for treatment planning. Ultimately, the skin platform can provide a physiologically representative high throughput system for understanding skin diseases and wounds and developing appropriate therapies. The relationship between the skin microenvironment, bacteria, and immune presence on wound evolution and resolution can provide key insights into treatment planning for wounds. This skin platform can be used to further screen drugs for skin infections or other conditions such as atopic dermatitis before proceeding to in vivo models and clinical trials and can easily be customized for any other type of infection, skin disease, or wound. The skin platform can also be used to assess dynamic transport of chemicals particularly toxicants, nanoparticles, or drugs and their response spatially in the skin.

In some embodiments, there is provided a model comprising a tumor with overlying skin, such as a model for breast cancer. In other embodiments, there are provide method for using lymphatic endothelial cells in the vascularized platform provided herein to create lymph blood vessels. These models can be used to study cancel cell invasion into overlying skin and surrounding lymphatics. The tissue may be surrounded by blood vessels and/or lymph vessels. The platform may be used to assess the effect of culture of different cells, drugs, or external stimuli, such as on vessel permeability.

In further embodiments, there is provided a provided an extracellular matrix comprising collagen and keratose. The matrix provided herein can be more thermally and mechanically stable as compared to previous extracellular matrix compositions. The matrix can be used in the platforms provided herein, such as for the growth of fibroblasts. The platform also allows for more accurate testing of temperature response and enables these platforms to respond to thermal insults or therapeutic heating with lasers, RF, and/or ultrasound similar to tissue.

The present disclosure is also directed to methods useful in the analysis of cell behavior. In an embodiment, a method is provided for the measurement of directed migration of cells, bacteria, and viruses in a microfluidic device. Generally a cell, bacteria, or virus is introduced into a first fluid-flow path and may attach to at least one of the surfaces in the flow path. The cell may also adhere to the scaffold. Either at the same time the cell is introduced into the first fluid-flow path or a different time, a biological entity may be introduced into the fluid-flow path. This biological entity may be a cell or sub-cellular component, including growth factors, cytokines, hormones, antibodies, gene expression and enzymes, as well as drugs and other small molecules. At a given time point, the extent of the migration of the cell, e.g., into the scaffold and optionally into the second fluid-flow path, is measured. The cell, bacteria, virus could also be introduced within the extracellular matrix or at the surface as in the case of skin and its transport and response in the platform measured.

In certain embodiments, the methods of the disclosure include the generation of multiple cell type biomaterials, useful in in vitro and in vivo systems such as tissue engineering. The devices described herein are used to fabricate biological or biocompatible materials that contain two or more types of eukaryotic cells. Further, in vitro systems described herein replicate the physiological functions of tissues or organ systems, and are thus useful in, for example, drug testing or toxicity screening of test compounds.

In the present studies, a multi tissue-on-a-chip platform was developed consisting of a vascularized breast tumor and healthy/tumorigenic liver microenvironments connected in series to enable dynamic determination of vessel permeability and transport of nanoparticles/drugs and their associated efficacy and toxicity to the liver. Microenvironments were fabricated from type I collagen of concentrations of 7 mg/ml and 4 mg/ml for tumor and liver respectively to replicate the growth characteristics and compression moduli of these tissues. Wall shear stresses of 4 dyn/cm² (healthy) and 1 dyn/cm² (tumor) were employed within each vessel to mimic physiological conditions. Cell morphology was characterized with immunofluorecent staining and the fidelity of liver cells cultured in the platform was demonstrated by measuring albumin release. Dextran particles with sizes of 3 kDa and 70 kDa were perfused in the platform to replicate the hydrodynamic diameters of chemotherapy drugs and nanoparticles conjugated with drugs. The platform was utilized to determine the effect of particle size on the dynamic and spatial diffusion of particles through each microenvironment independently and in response to circulation of particles in varying sequence of microenvironments (tumor to liver or liver to tumor). The results showed that when breast cancer cells were cultured in the microenvironments they had a 2.62-fold (p<0.001) higher vessel porosity compared to vessels within healthy liver microenvironments, which resulted in increased permeability of tumor microenvironment by 2.35- to 2.77-fold (p<0.01) for 3 and 70 kda particles, respectively, compared to healthy liver. Decreased particle accumulation of 2.57-fold (p<0.01) was observed for larger particles compared to smaller particles in the ECM of healthy liver. However, 5.57 (p<0.01) fold greater ECM accumulation of larger particles compared to smaller particles occurred for the breast tumor microenvironment. The particle accumulation within the breast tumor microenvironment decreased by 5.49-fold (p<0.01) if particles were first perfused through the liver and smaller particles demonstrated greater accumulation in the liver. Thus, the platform can be utilized to determine the impact of the tissue/tumor microenvironment or drug/nanoparticle properties on transport, efficacy, selectivity, and toxicity in a dynamic, and high throughput manner for use in treatment optimization.

The tissue on-a-chip microenvironment provided herein can be used to mimic transport in vivo enabling spatial and dynamic assessment of transport of any type of drug/nanoparticle as a function of their size. This device can be used to investigate the influence of other drug/nanoparticle properties including surface charge, dimensionality, targeting ligand, and aspect ratio on transport. By altering the direction of flow the effect of targeting and metabolism on transport kinetics of drugs/chemicals can be simulated in high throughput, inexpensive optimization of nanoparticles or other therapeutics by enabling toxicity, efficacy, and biodistribution measurements as a function of varying microenvironmental conditions and drug/nanoparticle properties. The multi tissue-on-a chip microenvironments can also be utilized for testing a combination of different treatment methods such as hyperthermia, radiation, and a myriad of nanoparticles with unique functionality to create solutions for targeted delivery.

By merging multiple embodiments of the platform including vascularized tumors and skin a first-of-its-kind physiologically representative three-dimensional comprehensive in vitro breast tumor platform for modeling invasion of aggressive breast cancer through the breast tissue, their interactions with nearby blood and lymphatic vessels, as well as invasion into the skin can be used to identify targetable stromal-tumor cell interactions driving the aggressive phenotype of breast tumors. The platform can be used to investigate the tumor stromal interactions of very unique but aggressive and metastatic breast tumors, modeled with SUM149, MDA-IBC3, and MDA-MB-231 cell lines. The platform can consist of functional vascular and lymphatic vessels as opposed to current existing platforms which include only nonfunctional vessels. The skin component will include the hypodermis, dermis, and epidermis. The platform can host stromal cells, MSCs, adipocytes, fibroblasts, keratinocytes, and macrophages, which have been shown to promote skin invasion behavior of breast tumors. The matrix can be composed of collagen ECM representative of breast tumor tissue without the presence of PDMS structural supports maintaining the in vivo tumor architecture. The platform can benefit multiple subtypes of aggressive breast cancer where tumor stromal interactions are also dominant in disease progression and enhance the study of migration phenotypes, both collective emboli migration as well as epithelial to mesenchymal transition.

### II. EXAMPLES

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1—Development and Characterization of Microfluidic Platform

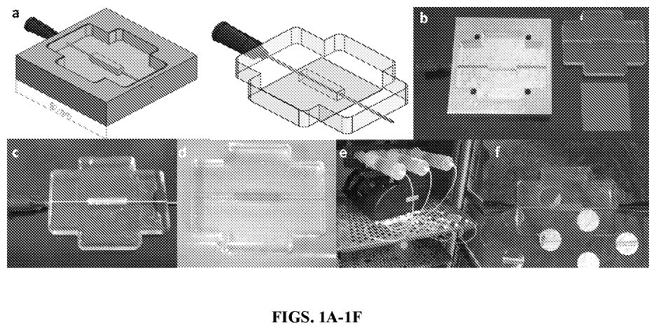

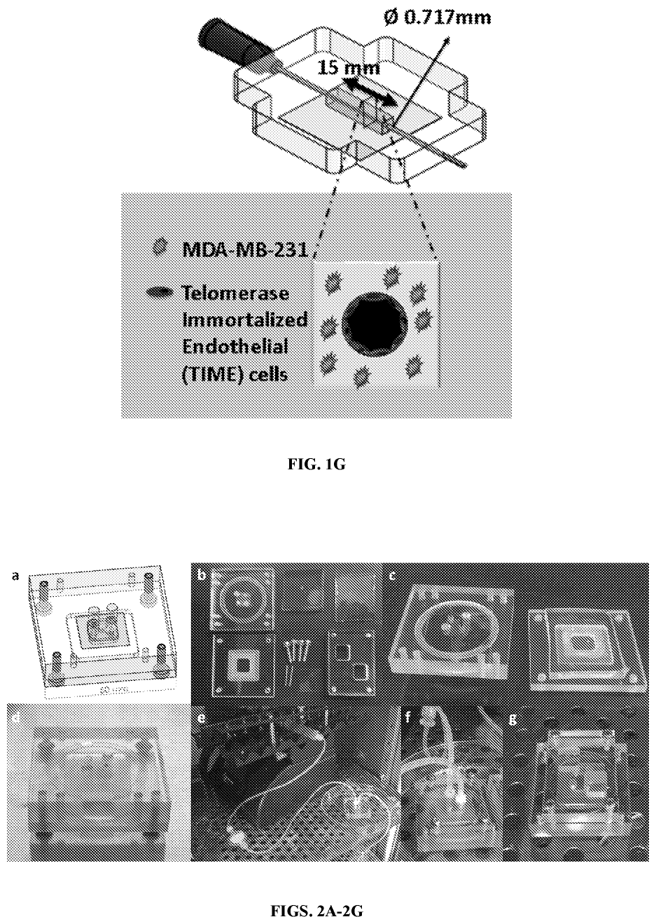

An in vitro platform was created as a single vessel vascularized tumor model to study the transport in a hyperpermeable tumor microenvironment resulting from tumor-vasculated interactions. The platform overcomes complications of restrictions to the geometry and size of the tissue chamber due to FEP tubing. The platform utilizes simplified fabrication techniques leading to increased reproducibility (FIG. 1) while maintaining endothelial integrity using flow preconditioning methods. The housing material mold for this platform is fabricated using micro-milling techniques. This decreases fabrication time and eliminates the requirement of clean room facilities, multistep fabrication processes, and expensive reagents to produce a vascularized microchannel. The platform can be easily customized to alter geometries of the tissue chamber and vessel size while maintaining a continuous lumen. Additional benefits include reduction in the amount of collagen and reagents required to fabricate the platform. The platform also establishes a simpler setup for live cell imaging of particle transport in the tumor. Dynamic co-culture of endothelial and tumor cells has produced an improved in vitro endothelialized microchannel with convenient setup for imaging and continuous observation.

A confluent endothelium was formed in the tumor microenvironment and then diffusion of particles through the endothelium was investigated. In addition, a microvascular network encased in a collagen hydrogel seeded with breast cancer cells created a physiologically representative vascularized tumor microenvironment (FIG. 6).

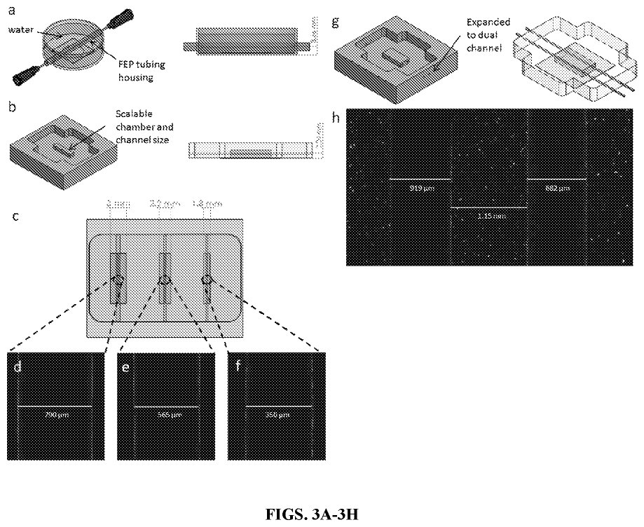

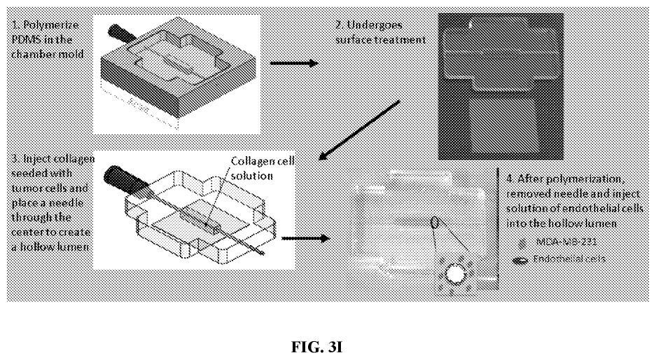

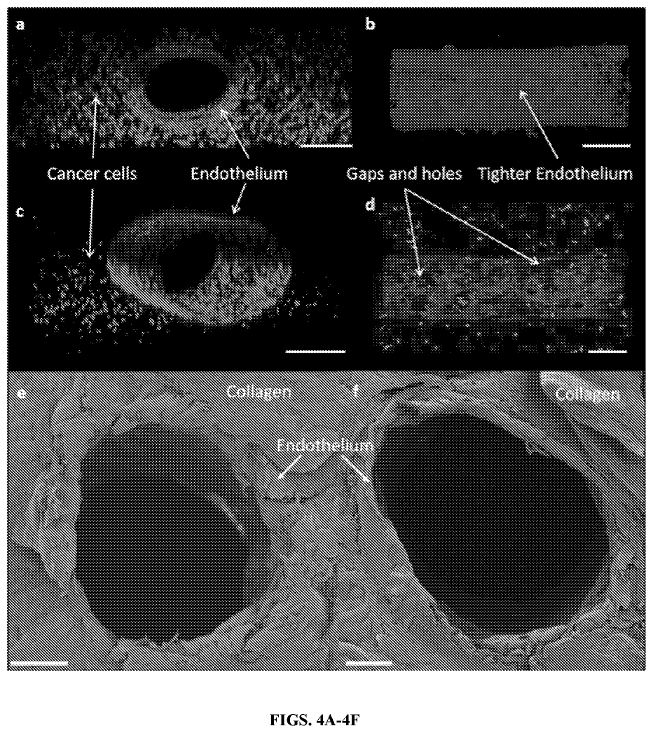

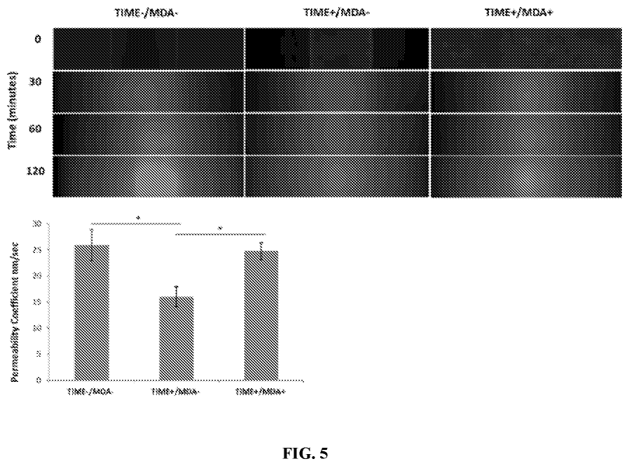

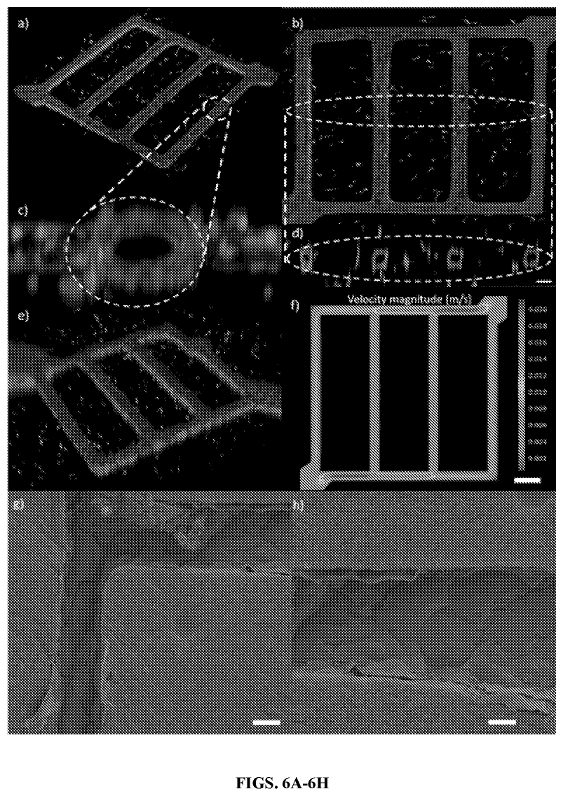

The platform has increased working distance and creates a viable in vitro tumor microenvironment that can interface with imaging setups to complete studies of the microenvironment. The bonding of a PDMS chamber directly to a glass cover slip allows the hydrogel to be positioned closer to microscope objectives and eliminates the necessity of using water to create an optically clear system for imaging. Water removal from the platform decreased the necessary microscope working distance by over a millimeter (FIG. 3A-B) and prevented potential fluid leaks while imaging. Inlet and outlet needles for flow are positioned more securely and promote a more stable platform for use on microscope stands. Using aluminum molds to form a PDMS tissue chamber enables scaling up of the tissue chamber and channel dimensions (FIG. 3B). Therefore, the platform size can be adjusted to create a series of other interconnected vascularized tissues in addition to a single tumor platform (FIG. 3C).

Linking different size platforms enabled creation of a simple body on a chip platform consisting of multiple chambers with unique vascularized tissue types. Endothelialized channels of different diameters were fabricated using 22, 25, and 30 gauge needles corresponding to vessel diameters of approximately 717, 514, and 311 μm respectively (FIGS. 3D-F). This allowed for a range of vessels sizes relevant to mammary tumor capillaries in the venous network to be mimicked. The single vessel platform was also expanded upon to create a dual-channel platform (FIG. 3G-H) capable of investigating the influence of pressure and biochemical gradient factors on tumor development and cell migration

Engineered Tumor Microenvironment:

The in vitro tumor platforms were composed of green fluorescent protein (GFP) labeled MDA-MB-231 cells encased in a collagen ECM with a confluent endothelium as shown in FIG. 4C. Following 72 hour flow preconditioning protocol, the TIME cells proliferated and elongated in the direction of flow as illustrated in FIG. 3B. The nominal outer diameter of a 22 gauge needle was 717 μm. After removal of the needle, preconditioning of cells, and sustained WSS during preconditioning, the final channel diameter could vary from the original of 717 μm to 900 μm. Collagen channels seeded with endothelial cells have been reported to expand to larger diameters after 3 days of preconditioning (Chrobak et al., 2006). In order to confirm the capability of the platform to maintain the vitality of cells, viability of the tumor cells was examined to determine if the bonding of a glass cover slip to the PDMS chamber created an oxygen deficient environment that does not support cell viability. The strong green fluorescence signal (live cells) with minimal red signal (dead cells) from both the endothelial cells and cancer cells in the collagen matrix revealed that the cells were viable and unaffected by being cultured and preconditioned in an enclosed PDMS chamber (FIG. 4A). SEM analysis of the endothelial channels shows that they remain patent following the 72 hour flow preconditioning protocol as shown in FIG. 4E-F.

The morphology of the endothelial layer for mono-culture and co-culture platforms was visualized using F-actin staining as evidenced by red fluorescence. Top view of the tissue chamber (FIG. 4) shows tight endothelium for the TIME mono-culture (FIG. 4B) with minimal holes in the cell layer; however, leakier endothelium with gaps between endothelial cell junctions was observed for the microchannel when endothelial cells were co-cultured with cancer cells as is also evidenced in vivo. The large gaps and holes throughout the endothelial layer occur due to the interactions between the cancer and endothelial cells. Previous studies have revealed that direct contact between endothelial and cancer cells decreases endothelial viability.

Transport: To add quantitative support to the confocal images showing endothelial behavioral in the single vessel platform, FIG. 4, the vessels permeability was measured. A progression of images flowing 70 kda dextran (260 μl/min) over a period of 2 hours shows an increase in the amount of dextran collecting in the hydrogel for each condition compared to the zero time point. During optimization of the experimental setup 2 hours was selected as a timeframe for allowance of a steady state permeability rate to be achieved. As expected, the acellular platforms without the presence of an endothelium exhibited the highest effective permeability coefficient average, 26±3 nm/sec with the most dextran transported into the bulk collagen, followed by the co-culture with an effective permeability coefficient of 25±1.7 nm/sec. Whereas the average value of the effective permeability coefficient for the mono-culture (endothelium only) was the lowest with the value of 16±1.9 nm/sec which results from the barrier function of the endothelium. The mono-culture platform was determined to have a significantly lower diffusion coefficient compared to both the co-culture and acellular platform (p<0.05). The intensity inside the channel itself does not change as the dextran is perfused under continuous flow. The higher intensity areas in the center of the 120 minutes (FIG. 5) result from the collection of light from dextran that has diffused through the bottom and top of the channel. The vessel permeability data re-enforces the conclusions from the f-actin confluency studies. The tight endothelium that forms in the mono-culture vascularized platforms serves to limit the amount of transport into the hydrogel. The inclusion of cancer cells creates a leaky endothelium (FIG. 4D) with pores allowing for increased transport out of the channel into the surrounding tissue resembling the well-known enhanced permeability effect. The extent of the leakiness formed in the co-culture platform prevents it from being significantly lower than acellular platform. The endothelium permeability properties influences the transport and effectiveness of particles and therapeutics into the targeted tumor site.

Others studies have reported similar results that the presence of tumor cells reduces the endothelial barrier function and increases transport of macromolecules through the endothelium into surrounding tissue (Buchnan et al., 2014). Introducing particles larger than dextran (>70 kDA) may decrease the diffusion rate through the endothelium and into the platform. Therefore, particles may congregate along the endothelial wall unable to pass through smaller gaps on the endothelium. Also, increasing the particle size may increase the difference between co-culture diffusion and a non-endothelialized channel, as the co-culture endothelium may be sufficiently confluent to prevent diffusion of larger sized particles.

Network Platforms:

In order to create an in vitro platform that better recapitulates the tumor microenvironment, a microfluidic vascular network was created that enabled improved perfusion and has the capability of scaling the platform to larger tumor sizes. Using soft lithography techniques, a simple geometric pattern was imprinted into a collagen hydrogel to form a network of channels encased in the platform. The network has one inlet and outlet to provide transport through the system. The width of each individual channel was approximately 100 μm (FIG. 6). Seeding TIME cells into the network and perfusion through the vessel resulted in a confluent endothelium throughout the network. By suspending MDA-MB-231 cells in the collagen, a 3D in vitro engineered microfluidic vascularized tumor was created as presented in FIG. 6A.

Evaluation of a top view of the co-culture network platform in FIG. 6B showed endothelial behavior similar to that of the single vessel platform in which the cells proliferate and elongate to form a confluent endothelium. The MDA-MB-231 cells interacted with the endothelial cells to create gaps forming leakier endothelium as evidenced by the endothelium in FIG. 6B. It was also observed that the use of collagen as a scaffold allowed the TIME cells in the channels to remodel their surroundings resulting in final geometries that deviate from the strict rectangular geometry of the PDMS stamp pattern that other groups are limited too. The corners at channel intersections develop a circular radius allowing for a continuous endothelium as opposed to the squared PDMS corners as shown FIG. 6C, D, G. Soft lithography patterns used for the formation of networks have a square cross section but it is evident from the images of the channel cross section (FIG. 6C), that the TIME cells remodel the surrounding ECM resulting in a rounded endothelium similar to in vivo vasculature. Fabrication of channels smaller than 100 μm diameter did not form a confluent endothelium as effectively through the entire network as the seeding of endothelial cells tended to clog the passageways. Similar findings have reported a minimum channel diameter of approximately 50 μm as the limit for this endothelialization process. The channels in the network remained patent (FIG. 6H) and capable of transport after the preconditioning needed to form the endothelial layer. This was verified by flowing blue 0.10 μm polymer microspheres through the network (FIG. 6E). Particle transport was tracked through the platform in real time and the even distribution was observed throughout the network. Moreover, expected velocity magnitude inside vasculature is provided in FIG. 6F. According to velocity magnitude inside vasculature provided by simulation results, the developed platform was able to provide sufficient pressure gradient to maintain physiological wall shear stresses, 1 dyn/cm², based on Poiselle flow and simulation prediction. Simulation results (FIG. 6F) demonstrated the capability to generate different velocity magnitudes in each vessel. Due to different velocity magnitudes in each vessel, WSS varies between 0.75-6.56 dyn/cm², which is within physiological WSS range reported in literature. Thus, verification of physiological flow conditions inside the vasculature showed that the developed in vitro platform successfully mimics in vivo tumor microenvironments.

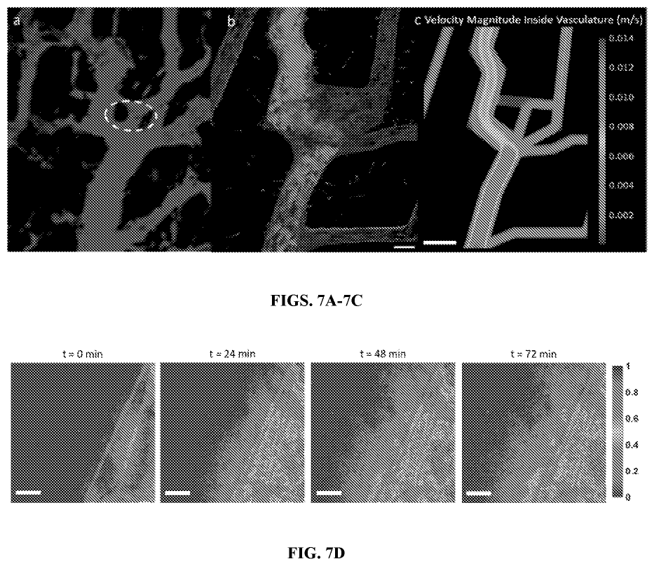

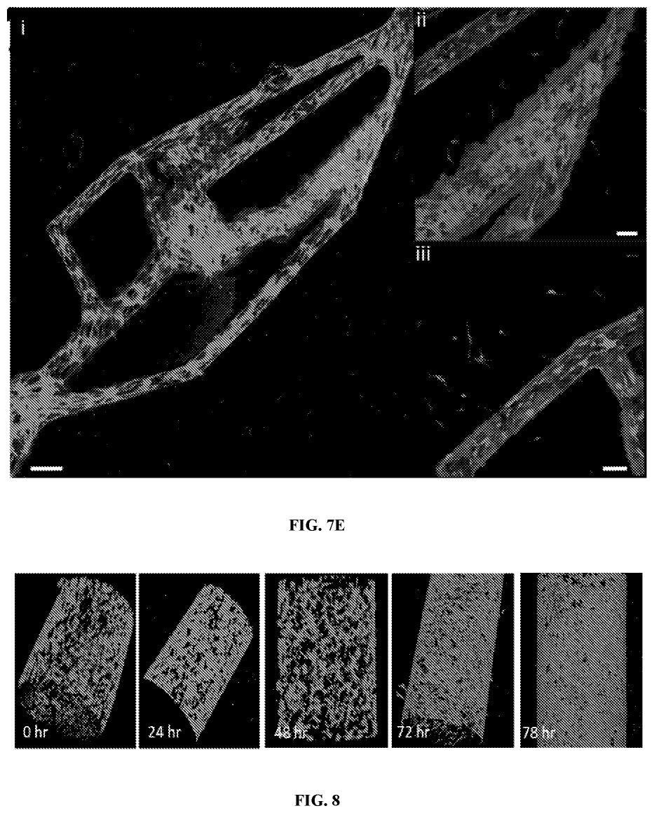

In vitro platforms that re-create physiologically relevant tumor vasculature tailored to an individual allows for evaluation and optimization of patient specific therapies. In these platforms transport of therapeutics could be studied in realistic environments and properties optimized to achieve targeted and efficient drug localization. Following the initial vascular network pattern presented in FIG. 6, a microfluidic network with tortuous geometry was fabricated to replicate vasculature patterns found in representative in vivo tumors. An image of in vivo tumor vasculature was selected as a model to recreate and test the capabilities of this additive tissue engineering method for creating complex geometries (FIG. 7A adapted from Tong et al., 2004). Limited work has been done to re-create patient specific geometries for in vivo studies. FIG. 7A produced by Tong et al., presents blood vessels injected with fluorescein-labeled dextran in a human colon adenocarcinoma LS174T tumor grown in severe combined immunodeficient mice and imaged using intravital microscopy. The in vitro platform presented in FIG. 7B mimicked the geometry of the vasculature. The design had one large channel with a width and depth of approximately 200 μm and multiple smaller branching channels with widths and depths of approximately 100 μm. Preconditioning of TIME cells in the channels remodeled the collagen to produce rounded corners and channel diameters of approximately 200 and 100 μm.

FIG. 7C shows FEA simulation results of velocity magnitude inside the in vivo vascular patterned platform with WSS varying between 0.75-2.55 dyn/cm². Similar to network platform, simulation results show physiological shear stress inside the in vivo vascular patterned platform. While this fabrication technique is capable of creating networks with complex geometries and multiple microchannel mimicking in vivo cases, it does present certain limitations. Lithography techniques can be used to create complex and detailed patterns; however, transferring these patterns to a collagen hydrogel is challenging. In order to capture the tortuous geometry in FIG. 7, a lower, less viscous, collagen concentration of 6 mg/mL was used. Using lower collagen concentration increased the reproducibility of the hydrogel's ability to better encase the tortuous geometries of the PDMS pattern but was still unable to capture the smallest spatial geometries. Another limitation of using lithographic techniques is that there is no means to create a gradual transition from larger to smaller channels, i.e. 100 μm channels connecting to 200 μm channel at a blunt interface. However, despite this interface, cells were capable of forming a confluent endothelium which is an important factor for transport studies. Also, increasing the number of microchannels with varying diameters quickly elevates the complexity of fabrication. Advances in 3D bioprinting are an alternative method for improved and fast fabrication of customizable advanced networks that move beyond the planar limitations of lithography.

For the first time, transport of two different sized particles in an in vivo vascular patterned platform was investigated and presented in FIGS. 7D-E. FIG. 7D illustrates transport of 70 kDa green fluorescent dextran, a common marker for protein and drug permeability, through the platform and diffusion into the surrounding ECM. Tumor region in between multiple vessels is saturated with dextran within the 30 minutes of perfusion but it takes over an hour to penetrate further into the collagen ECM. This transport behavior is representative of drugs and chemotherapies having a harder time penetrating into the dense tumor ECM and reaching cells farther from the blood vessels in vivo, especially for abnormal, heterogeneously distributed tumor networks as demonstrated in this study. In addition to dextran, the platform was also perfused with 0.10 μm blue particles as shown in FIG. 7E. As the particle travel through the platform, they aggregate at the boundaries of the endothelium. As evidenced in FIGS. 7D-E, while vessel leakiness can influence transport of small molecules like dextran, transport of larger particles is dependent on additional parameters.

The present disclosure presents devices and methods for the fabrication of microfluidic channels by employing additive and subtractive tissue engineering techniques. Collagen scaffold was used in platforms to accommodate cells culturing and remodeling producing endothelialized vessels of in vitro tumor microenvironments. Vascularized tumor platforms were created with embodiments of scalable channels (single/dual) and networks based upon in vivo vasculature. The single channel platforms allowed the dynamic tracking of tumor-vasculature interactions as well as the spatiotemporal behavior of particle diffusion in a physiologically representative microenvironment. The network platforms can be designed to replicate patient data and allow for the study of transport and drug delivery in conditions that mimic the patient's tissue. All these platforms can be expanded upon to incorporate immune cells, stromal cells, and lymphatic vessels to create a complete tumor microenvironment and be used to evaluate the toxicity of chemotherapeutics and lead to the development of new therapies.

### Example 2—Materials and Methods

Materials:

Stock solutions of collagen type I (14 mg mL⁻¹) derived from rat tails were prepared. Platforms of varying complexity ranging from single vessel platforms to vascular network platforms were fabricated. Single vessel platforms recreated the tumor endothelial microenvironment of a single blood vessel whereas the vascular networks represented branching blood vessels present in a tumor. Single vessel platforms were formed from polycarbonate and polydimethylsiloxane (PDMS) housing components that interfaced with glass cover slips to produce a tissue chamber with an imaging surface as shown in FIG. 1A and FIG. 2A. Stainless steel 4-40 machine screws fixed the computer numerical controlled (CNC) machined polycarbonate network platform components together (FIG. 2D). Machined aluminum molds were used to form PDMS components for single vessel platforms (FIG. 1A-B). Chamber surfaces were treated with 1% polyethyleneimine (PEI) in dH₂O and 0.1% glutaraldehyde in dH₂O. Standard soft lithographic techniques were used to produce patterned PDMS stamps for use in forming network platforms. PDMS was mixed at standard formation of 1 part catalyst to 10 parts base and cured for a minimum of 1 hour at 70° C. Human breast carcinoma (MDA-MB-231) cells were purchased from American Type Culture Collection (ATCC). Telomerase-immortalized microvascular endothelial (TIME) cells were obtained from Dr. Shay Soker of Wake Forest Institute for Regenerative Medicine. Cell culture media was purchased from Lonza. Oregon Green dextran 70kDA (Life Technologies) was used for transport studies. Preparation and use of all components and solutions was performed under sterile conditions.

Platform Design and Fabrication: All polycarbonate and aluminum components used in fabricating the hydrogel scaffolds were CNC machined. The aluminum mold used for the single vessel platform holds a 22G needle and was used to form a PDMS chamber (FIG. 1A) with an inlet and outlet for flow. All components were cleaned with 70% ethanol for sterilization and dried before use. To bond the PDMS chamber and glass cover slip together the individual components were plasma treated for 4 minutes (Harrick Plasma) before being assembled. After assembly, to increase adhesion of collagen, the tissue chamber surface was treated with PEI for 10 minutes followed by glutaraldehyde for 20 minutes and then rinsed with dH₂O leaving the platform ready for the formation of collagen hydrogels. For the network platforms, stainless steel 4-40 machine screws fixed the CNC machined polycarbonate network platform components together. For the network platform the polycarbonate components, glass cover slip, and pins received the same sterilization treatment (FIG. 2B). The tissue chamber wells in the top and bottom polycarbonate components received the same surface treatments. The polycarbonate components were fixed together using screws to form the tissue chamber. Standard soft lithographic techniques were used to produce patterned PDMS stamps for use in forming network platforms. For both single and network platforms, PDMS chamber and glass cover slip were bonded together by exposure to plasma treatment before being assembled.

Formation of Collagen Hydrogels: A working collagen solution (6-7 mg mL⁻¹) for use in the platforms was prepared by neutralizing the stock collagen solution. Stock collagen was mixed over ice with 10×DMEM, 1N NaOH, and 1×DMEM. This solution was added to the single vessel channel or vascular network platform chambers and polymerized in an incubator for 25 minutes at 37° C. For the single vessel platform a 22G needle was left encased in the collagen during the polymerization process (FIG. 1C). After removing the needle a cylindrical channel with a diameter of approximately 717 μm extending the length of the hydrogel remained (FIG. 1D).

For the vascular network platform, working collagen was prepared in the same manner; the well in the base component of the platform was filled with collagen. Then, a flat PDMS piece was laid on top of the well to produce a flat collagen surface after polymerization (FIG. 2C). The well in the top component was aligned with a lithographically produced PDMS stamp that had the designed channel pattern, and pins were inserted into the chamber to create an inlet and exit port before the chamber was filled with collagen and polymerized (FIG. 2C). After polymerization and removal of the PDMS and pins, the top and bottom components of the platform were stacked resulting in a network fully encased in collagen (FIG. 2D) (Zheng et al., 2012; incorporated herein by reference). The process of forming biomaterials about a lithographic pattern produces a square cross-section in the channel. Once cells are seeded in the channel and a confluent endothelium is established a circular cross-section is formed. The average diameter of tumor microvessels has been reported to be less than 100 μm. However, it has been reported that seeding channels with a diameter of approximately 50 μm with endothelial cells presents issues as the cells tend to aggregate and clog the channels (Tien, 2014). Based on this information our stamps were designed to have diameters of 100 μm or greater.

Cell Culture in Gels: To create a tumor microenvironment, cancer cells were suspended in the working collagen solution to the targeted density of 1×10⁶ cells/mL (Buchanan et al., 2014). The collagen solution containing cells was injected in the chamber followed by incubation of the platform chamber at 37° C. for 25 minutes. After polymerization of the collagen and the formation of vascular channels in the scaffold, TIME cells were injected into the microchannels. For the single vessel platforms, TIME cells (2×10⁷ cells/mL) were injected twice at 10 minute intervals and the platform was slowly rotated during the intervals to promote cell adhesion around the entire channel. To seed endothelial cells into the network platforms 15 μL of media containing TIME cells (5×10⁶ cells/mL) was added to the inlet and allowed to perfuse into the channels for 20 minutes at 37° C. The MDA-MB-231 cells expressed a green fluorescent protein (GFP) signal and TIME cells a red fluorescent protein (RFP) signal.

Formation of Endothelialized Channels:

To form a confluent endothelium the platforms were connected to a syringe pump (Harvard Apparatus) providing a continuous flow of TIME cell media into the channels resulting in a shear stress (τ) of 0.01 dyn/cm² for 36 hours followed by 36 hours of 0.1 dyn/cm² (FIG. 1E-F and FIG. 2E-F). The desired flow rate in the channels was calculated from a target shear stress τ assuming Poiseille flow:

$$τ = \frac{4 Q μ}{π {r}^{3}} \qquad ( 1 )$$

where Q is the volumetric flow rate, μ is the dynamic fluid viscosity, and r is the radius of the channel. Poiseille flow in the channel was confirmed using μ-PIV. This preconditioning protocol has been previously established by the Rylander group to produce a confluent and aligned endothelium (Buchanan et al., 2014). Air eliminating filters were placed upstream of the inlet to prevent bubbles from entering the channels during perfusion. The outlets fed to a collection reservoir.

An alternate method was also developed to provide flow for the vascular network platform that uses a simplified setup of a reservoir system. The simplified set up is a quicker and easier method to establish flow and operates based on gravity creating a pressure difference at the inlet and outlet to induce a flow through the system (FIG. 2G). A syringe pump driven flow or the alternate gravity driven flow was used to precondition the network platform. The gravity reservoir method was used regularly as it had a simpler setup. Using the gravity reservoir preconditioning method required establishing a height difference of approximately 6 mm between the inlet and outlet reservoirs providing a decaying flow rate. The height difference was reestablished every 12 hours over 48 hours preconditioning period.

Viability Analysis:

In order to confirm the utility of the platform the viability was assessed. For this measurement, untagged MDA-MB-231 cells were incorporated in the collagen to form the tumor microenvironment. The viability of MDA-MB-231 cells in the tumor platforms was evaluated using calcein AM (live)/propidium iodine (PI) (dead) (ThermoFisher Scientific) corresponding to green and red stains, respectively (FIG. 4A). After completion of preconditioning the platform, the MDA-MB-231 cells in the collagen were incubated in 1 μM Calcein AM solution for 30 minutes followed by a 10 minute incubation in 45 μM PI solution. Samples were imaged immediately after PI treatment using Leica TCS SP8 confocal laser scanning microscope with HC PL Fluotar 10×/0.30 objective.

Endothelium Morphology:

F-actin staining and SEM analysis was completed immediately following endothelial preconditioning to visualize endothelial morphology and orientation in the microchannels. For F-actin staining, the microchannels were perfused with 4% paraformaldehyde and 0.5% triton-X-100 (Sigma Aldrich) for 20 minutes followed by incubation in 1% bovine serum albumin for 30 minutes. Rhodamine Phalloidin (ThermoFisher Scientific) probe was used to label F-actin and DAPI was used to label nuclei and imaged using Leica TCS SP8 confocal laser scanning microscope with HC PL Fluotar 10×/0.30 objective. SEM analysis was performed using a Zeiss Supra40 SEM-Electron Microscope. Microchannels were fixed overnight in an aldehyde mixture osmium treatment for 4 hours. Post fixation, the platforms were dehydrated in a series of ethanol solutions (50-70-95%), then critical point dried by CO₂ and coated with a thin layer of platinum-paladium.

Permeability:

Permeability coefficients were obtained to quantify the rate of transport through the endothelium of the microchannel in response to perfusion of 70 kDa dexran particles. Particles with a molecular weight of 70 kDA are commonly used in transport studies and is comparable to large macromolecules. Three conditions of the single vessel platform were evaluated including an acellular microchannel, an endothelialized microchannel without cancer cells in surrounding collagen, and a microchannel containing co-culture of endothelial cells in vessels with cancer cells cultured in the surrounding collagen. Studies were conducted upon completion of the 72 hour preconditioning protocol. Green fluorescent dextran suspended in serum free endothelial basal media (EBM-2) at 10 μg/mL was perfused through the microchannels for 2 hours at a flow rate of 260 μL/min generating a WSS of 1 dyn/cm² with images being taken every five minutes for evaluation of transport and diffusion. In normal microvessels the average WSS is around 4 dyn/cm². The abnormal vasculature in tumors can compromise flow resulting in reduced WSS relative to normal blood capillaries and this led to the selection of a lower value of 1 dyn/cm² for use in particle transport studies.

Imaging for diffusion studies was completed on a widefield inverted Leica DMI 6000 B fluorescence microscope and the tiff images obtained were exported to MATLAB for evaluation. The average fluorescent intensity of the diameter of the collagen was measured and used to determine the diffusion permeability coefficient Pa. This coefficient describes the ability of solute to pass uniformly from the microchannel into the surrounding hydrogel and is calculated with the following equation:

$${P}_{d} = \frac{1}{{I}_{1} - {I}_{b}} ( \frac{{I}_{2} - {I}_{1}}{Δ t} ) \frac{d}{4} \qquad ( 2 )$$

where I_{b} is the background intensity, I₁ is the average initial intensity, I₂ is the average intensity after recovery time interval Δt, and d is the diameter of the microchannel (Price et al., 2011). The last five consecutive data points from the 2 hours of flow were used to calculate Pa as these were the time points at which stable and consistent measurements were achieved. Three samples (n=3) were collected for each variation in the diffusion studies. Data is expressed as a mean value ±standard deviation. Significance of the data was verified using Student's t-test and a 95% confidence criteria between groups of data. Blue 0.10 μm fluorescent polymer microspheres were used to visualize particle transport in the network tumor platform to contrast against alternate colors of labeled cancer and endothelial cells. The microspheres were suspended in serum free endothelial media at a density of 10 μg/mL and allowed to perfuse into the system for 1 hour.

Modeling Flow Inside the Vasculature:

WSS is one of the key factors in creating physiological in vitro tumor microenvironments. In order to ensure that continuous flow is sustained throughout the microfluidic platforms, velocity profiles were modeled using Comsol Multiphysics. Stokes law was used to quantify the flow inside the vasculature with the following assumptions: constant fluid viscosity, incompressible fluid, and a low Reynold's number inside the vasculature:

$$ρ \frac{\partial u}{\partial t} = ∇ P + μ {∇}^{2} u \qquad ( 3 )$$

where ∇ is gradient operator and ∇² is the square of vector Laplacian, P is pressure, u is velocity, μ is viscosity and ρ is density of the fluid.

Flow inside a porous tissue such as the ECM must be modeled. The porosity has a significant impact on flow inside porous membranes and necessitates the use of Darcy's Law, which predicts transport in porous material:

$$u = - \frac{κ}{μ} ∇ P \qquad ( 4 )$$

where, κ is the permeability of the material, P is the pressure, u is the velocity and μ is the viscosity. Mass conservation (Equation 5) is also solved simultaneously.

∇·u=0 (5)

Simulations were run on the network platforms with the following conditions: constant flow rate of 260 μl/min, and inlet and outlet boundary conditions of zero gauge pressure. Other ECM properties used in the simulation were permeability of collagen of 10×10⁻¹⁵ m² and porosity of collagen of 0.49. Resulting flow velocity profile was used to calculate WSS at the vessel walls. WSS of a Newtonian fluid is defined as:

$${τ = μ \frac{\partial u}{\partial y} \vert}_{wall} \qquad ( 6 )$$

where u is velocity parallel to the vessel, and y is the perpendicular direction to the wall.

### Example 3—Breast Tumor Model

A 3D in vitro vascularized tumor platform was developed as a tool for modeling various types of aggressive breast cancer where tumor stromal interactions including tumor-vasculature and tumor-ECM interactions have been shown to direct the disease phenotype. The 3D in vitro vascularized platform was utilized to model both IBC tumors as well as non IBC invasive ductal carcinoma showing the versatility of the platform for studying a wide variety of breast cancers. It replicates conditions that are representative of in vivo tumor vasculature interface such as physiological flow and associated shear stress, a continuous, aligned and functional endothelium while allowing for tumor-endothelial-ECM interactions. The tumor cells of interest were the IBC cells lines SUM149 and MDA-IBC3, and non-IBC invasive ductal carcinoma cell line MDA-MB-231. All these cells lines are hormone receptor negative which correlates with breast cancer of high malignancy and recurrence, and additionally, SUM149 and MDA-MB-231 cells are triple negative indicating they lack amplification of the HER2 receptor. The influence of paracrine signaling was investigated between tumor cells and the vascular endothelium and the in vivo response of a hyperpermeable tumor vasculature was recreated as well as secretion of proangiogenic cytokine VEGF. ECM porosity was characterized as a function of the interactions between the tumor cells and the stroma which revealed a response similar to in vivo migratory behavior of tumor cells. These platforms provide a tool to elucidate disease dynamics of aggressive breast cancer tumors where tumor-stroma interactions are the driving force behind tumor development and progression.

In Vitro 3D Tumor Platform:

The in vitro vascularized tumor platforms consisting of co-culture of either IBC or non-IBC cancer cells with TIME cells was developed. The 78-hour flow protocol with a graded increase in WSS from 0.01 dyne/cm² to 1 dyne/cm² resulted in a confluent endothelium as shown in FIG. 8. FIG. 8 shows the evolution of the endothelium with the cells aligning and creating a tight confluent barrier with increasing time. The resulting endothelium serves as the baseline upon which to evaluate the influence of different cancer cells, IBC and non-IBC, on the surrounding vessel with respect to endothelial morphology and secretion of protumor cytokines.

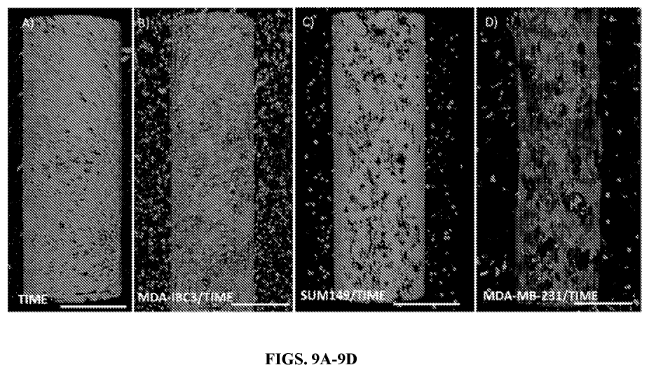

Following the 78-hour graded flow treatment to establish a confluent endothelium, the resulting in vitro vascularized co-culture platforms are shown in FIG. 9. SUM149 is a highly invasive triple negative IBC cell while MDA-IBC3 is negative for hormone receptors, but overexpress HER2 (human epidermal factor) receptor. Studies have shown SUM149 cells to display high tumorigenic behavior with formation of both local and distant metastases and aggressive skin invasion while MDA-IBC3 formed local metastasis along with skin invasion. MDA-MB-231, similar to SUM149 is a highly migratory and invasive triple negative breast cancer cell line. FIG. 9 reveals the feasibility of the platform to model various aggressive breast cancers including MDA-MB-231, SUM149 and MDA-IBC3, all breast cancer cell lines whose phenotype has shown to be influenced by their interactions with the tumor stroma.

Endothelium Integrity:

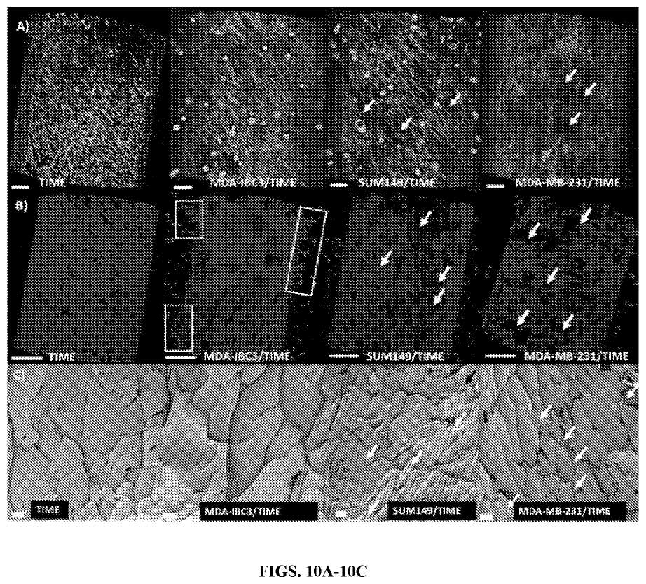

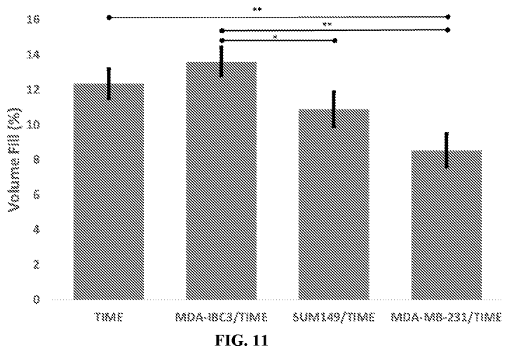

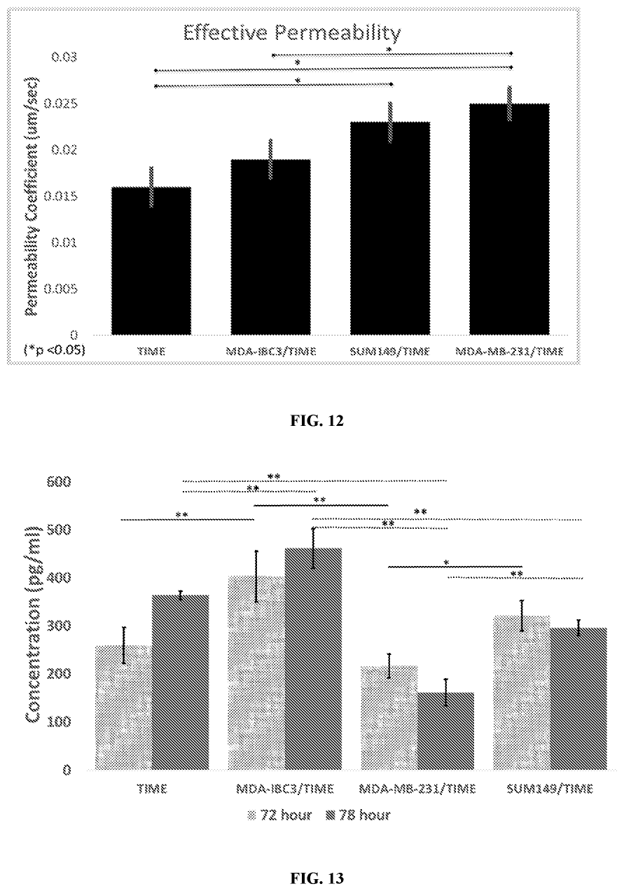

Hyperpermeability and leakiness of tumor blood vessels is characteristic of in vivo breast cancers and studies have revealed this to be influenced by tumor-vasculature interactions. FIGS. 10 and 11 reveal the formation of a leaky vasculature in the in vitro vascularized platforms of SUM149/TIME and MDA-MB-231/TIME representative of the in vivo behavior of tumor blood vessels. Co-culture of TIME cells with the triple negative breast cancer cells MDA-MB-231 and SUM149 resulted in a patchy endothelium as seen by the presence of black voids in the endothelium compared to MDA-IBC3 and TIME only in vitro vascularized platforms as evidenced in FIG. 9. Endothelium integrity and morphology was investigated by staining for PECAM-1 or platelet endothelial cell adhesion molecule-1 and F-actin as well as SEM analysis as shown in FIG. 10. Stained images of both PECAM-1 and F-actin in FIG. 10A-B shows a disturbed and leaky endothelium caused by the presence of SUM149 and MDA-MB-231 as denoted by the white arrows indicating some of the voids and gaps in the endothelial lumen.

Staining patterns of PECAM-1 in FIG. 10A revealed a bright fluorescent signal present uniformly across a majority of the endothelium in the TIME and MDA-IBC3/TIME in vitro vascularized platforms whereas PECAM-1 expression in SUM149/TIME and MDA-MB-231/TIME is less uniform and more dispersed. While the triple negative breast cancer cells SUM149 and MDA-MB-231 resulted in a leaky endothelium, presence of MDA-IBC3 cells in contrast to SUM149 displayed early signs of angiogenic sprouting with TIME cells starting to bud from the borders of the endothelial channel (boxed areas in FIG. 10B) towards MDA-IBC3 cells replicating another phenomenon found in vivo IBC tumors. SEM analysis of the endothelium provided high resolution images revealing endothelial adhesion to collagen matrix and their morphology. As seen in FIG. 10C, the adhesion of TIME cells to collagen matrix follows patterns seen in the immunofluorescent staining results as well as in vivo response. Endothelial cells in the TIME and the MDA-IBC3/TIME platforms show a tight endothelium with the endothelial cells spread out and overlapping in contrast to SUM149/TIME and MDA-MB-231/TIME platforms which showed empty voids between adjacent endothelial cells as noted by the white arrows.

Comparison of endothelial coverage of the lumen showed a significant decrease in the endothelium coverage in the triple negative breast cancer platforms of SUM149/TIME and MDA-MB-231/TIME in comparison to MDA-IBC3/TIME and TIME in vitro vascularized platforms as illustrated in FIG. 11. SUM149/TIME showed a 1.25-fold decrease in coverage compared to MDA-IBC3/TIME in vitro vascularized platform while MDA-MB-231/TIME had a 1.6-fold and 1.5 decrease in volume fill compared to MDA-IBC3/TIME and TIME only in vitro vascularized platforms.

Vascular Permeability:

Intravasation and extravasation of tumor cells for metastasis as well as inefficient delivery of drugs and chemotherapeutics to the tumor are highly influenced by vascular permeability and leakiness. To determine the effect of tumor cells on transport across the endothelium barrier, the platforms were perfused with fluorescently labeled dextran and the resulting changes in florescent intensity from the dextran perfusing across the endothelium were used to determine the effective permeability (FIG. 11). The measured effective permeability for TIME, MDA-IBC3/TIME, SUM149/TIME, and MDA-MB-231/TIME were 0.016±0.002, 0.019±0.002, 0.023±0.002, 0.025±0.002, respectively. Vascular permeability of the MDA-MB-231/TIME in vitro vascularized platform was 1.6- and 1.3-fold higher than TIME only and MDA-IBC3/TIME in vitro vascularized platforms while the permeability of the SUM149/TIME in vitro vascularized platform was 1.4-fold higher than the TIME only.

VEGF ELISA:

ELISA measurements for VEGF were performed on flow media collected at 72 hours (following formation of the baseline endothelium) and 78 hours (after exposure to WSS of 1 dyn/cm² for 6 hrs) as illustrated in FIG. 13. VEGF expression was significantly higher at both time points in MDA-IBC3/TIME in vitro vascularized platforms. VEGF expression in the MDA-IBC3/TIME in vitro vascularized platform was 1.6- and 2-fold higher at the 72 hour time point and 1.3- and 3-fold higher at the 78 hour time point compared to TIME only and MDA-MB-231/TIME in vitro vascularized platforms. Additionally, there was a similar trend of decrease in the VEGF expression in the triple negative leaky in vitro vascularized platforms while the more confluent platforms of MDA-IBC3/TIME and TIME showed an increase in the VEGF expression at the 78 hour time point compared to the 72 hour time point.

Matrix Porosity:

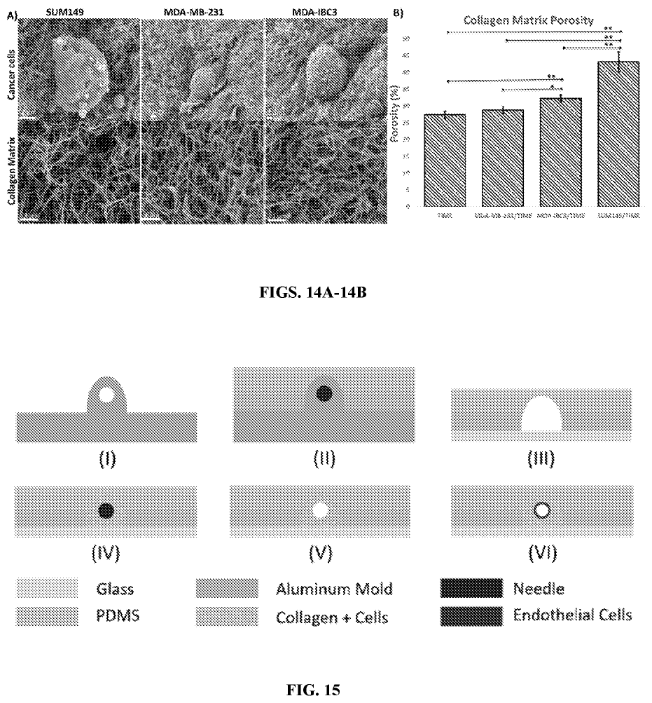

Invasive tumors modulate the surrounding ECM in order to migrate through the tumor microenvironment and out into the surrounding tissue. To quantify the ability of the tumor cells to modulate the ECM, SEM analysis of the collagen matrix in each of the vascularized breast tumor platforms was used to determine matrix porosity as shown in FIG. 14. FIG. 14A revealed the difference in morphology of the various tumor cells with the SUM149 and MDA-IBC3 cells showing a more rounded phenotype while the MDA-MB-231 displays a mesenchymal like phenotype replicating behavior found in vivo. Porosity measurements depicted in FIG. 14B revealed both the IBC in vitro vascularized tumor platforms having a significantly more porous ECM compared to MDA-MB-231/TIME and TIME in vitro vascularized platforms. SUM149/TIME in vitro vascularized platform was 1.6, 1.5 and 1.3-fold higher in matrix porosity compared to TIME only, MDA-MB-231/TIME and MDA-IBC3/TIME in vitro vascularized platforms respectively. MDA-IBC3 in vitro platforms also showed a significant increase in porosity compared to the MDA-MB-231 and TIME only platforms. It is important to note that these values are relative measurements due to glutaraldehyde and critical point drying treatments necessary for SEM sample preparations.

Thus, a 3D in vitro vascularized tumor platform is provided herein to model the interactions of multiple aggressive breast tumor with their corresponding stroma and reproduced the in vivo response in terms of vascular permeability and ECM degradation. Utilizing the vascularized breast tumor platforms, it was shown that the type of tumor cell present had a profound impact on the endothelium with the more aggressive and invasive tumor cells creating a leakier vasculature and have provided the first opportunity to spatially observe and quantify this difference. Additionally, it was revealed that the co-culture of tumor and endothelial cells influences the expression levels of the angiogenic cytokine VEGF as well as remodeling of the collagen ECM. Modulation of the ECM and the endothelium in breast cancer are important factors that have been linked to tumor angiogenesis and metastasis and are influential parameters when studying tumor development and progression.

The 3D in vitro vascularized platforms provided herein allow for the investigation and modeling of the tumor-stroma interactions and the influence of the interactions on vascular permeability and matrix porosity for three different highly invasive and aggressive breast cancer phenotypes. Blood vessel leakiness and increased matrix porosity that are representative of in vivo behavior or invasive tumors were recreated. Compared to current 3D in vitro tumor models that focus on recreating specific stages of tumor progression, the platforms provided herein were able to model various stages in cancer progression including early signs of angiogenesis as well as modulation of tumor ECM and vasculature for migration and metastasis. The models can be used as a tool for studying various aggressive breast cancers whose phenotype is driven by tumor stromal interactions. While these platforms do not encompass the entire complexity of the tumor microenvironment, they provide an initial insight into the behavior of aggressive tumors and can be adapted to include other stromal components.

### Example 4—Materials and Methods

Cell Culture: Human breast carcinoma cell line MDA-MB-231(ATCC® HTB-26™) breast carcinoma, human breast inflammatory cancer cells MDA-IBC3 and SUM149, and telomerase-immortalized human microvascular endothelial (TIME) cells were used in this study. A lentiviral vector system was used to genetically modify MDA-MB-231 and TIME to stably produce either a green fluorescent protein (GFP) or a red fluorescent protein (RFP) respectively for real time imaging studies. Stable fluorescent MDA-MB-231 and TIME cells were from Dr. Shay Soker at the Wake Forest Institute for Regenerative Medicine (Winston-Salem, N.C.). MDA-IBC3 and SUM149 IBC cell lines labeled with GFP were from Dr. Wendy Woodward at MD Anderson Cancer Center (Houston, Tex.).

MDA-MB-231 cells were cultured in Dulbecco's Modified Eagle's medium, nutrient mixture F-12 (DMEM/F12) (Sigma Aldrich) supplemented with 1% penicillin-streptomycin (P/S) (Invitrogen), and 10% fetal bovine serum (FBS). MDA-IBC3 and SUM149 cells were cultured in Ham's F-12 media supplemented with 10% FBS, 1% antibiotic-antimycotic, 1 μg/ml hydrocortisone, and 5 μg/ml insulin. TIME cells were cultured in EBM-2 endothelial growth media supplemented with a growth factor BulletKit (Lonza CC-4176). All cell cultures utilized in this study were maintained in a 5% CO₂ atmosphere at 37° C. in an incubator.

In Vitro 3D Tumor Platform Fabrication:

The in vitro 3D tumor microfluidic platforms utilized in this study were composed of collagen type I matrix seeded with either MDA-MB-231, MDA-IBC3, or SUM 149 with a hollow channel seeded with RFP labeled TIME cells housed in a PDMS scaffold. Collagen type I extracted from rat tails was prepared following published protocols to produce stock collagen concentration of 14 mg/ml which was then neutralized with a solution consisting of 10×DMEM, 1N NaOH, and 1×DMEM to produce a final collagen concentration of 7 mg/ml. GFP labeled IBC and non-IBC cells were seeded at a density of 1×10⁶ cells/mL in the 7 mg/ml neutralized collagen solution and polymerized around a needle at 37° C. for 25 minutes. After polymerization, the needle was removed and the resulting hollow void was filled with a solution of 2×10⁵ TIME cells to form an endothelialized vessel lumen. Once TIME cells have had sufficient time to attach to the collagen matrix, flow was introduced using a syringe pump system. A 72 hour graded flow protocol was used to establish a confluent endothelium followed as we have previously published. Briefly, flow was perfused to expose the endothelium to wall shear stress (WSS) (τ) of 0.01 dyn/cm² for 36 hours followed by 36 hours of 0.1 dyn/cm². After completion of the 72 hour graded flow protocols, the in vitro vascularized platforms were exposed to 1 dyn/cm² for 6 hours.

Immunofluorescent Staining:

Endothelial morphology and cell-cell junctions were analyzed by performing immunofluorescent staining for PECAM-1 and F-actin upon completion of the 78 hour graded flow protocol. The staining protocol consisted of perfusing the platforms with 4% paraformaldehyde and 0.5% triton-X for fixation and permeabilization of the cell membranes, respectively. Next, the platforms were incubated in 5% BSA followed by overnight incubation with antibodies for PECAM-1 (Abcam, ab215911) and Rhodamine Phalloidin (ThermoFisher, R415).

Endothelial Permeability:

Endothelial vessel permeability as a function of paracrine signaling between tumor and vasculature was determined by perfusing the channels with 70 kDa GFP labeled dextran. Four conditions of the 3D in vitro vascularized tumor platforms were tested: TIME cell only platform, and platforms consisting of co-culture of TIME cells with either MDA-MB-231, MDA-IBC3, or SUM149 cells. After completion of the flow protocol for establishing a confluent endothelium, green fluorescent dextran suspended in serum free media (10 μg/ml) was perfused through the platforms with images taken every five minutes. The average fluorescent intensity was measured from the images and used to determine the diffusion permeability coefficient Pa as previously published (Buchanan et al., 2014). Three samples (n=3) were used for each platform condition with the resulting permeability factor expressed as a mean value ±standard deviation. Significance of the data was verified using one-way ANOVA and a 95% confidence criteria.

Enzyme-Linked Immunosorbent Assay:

Expression of VEGF was measured using enzyme-linked immunosorbent assays (ELISA) at two points: upon completion of the graded flow protocol (72 hours) for establishing a confluent endothelium and after exposure to WSS of 1 dyn/cm² (78 hours). 1 ml samples of perfusion media were collected from the flow outlet and ELISA was performed as per manufacturer's protocol (R&D Systems, DVE00).

Scanning Electron Microscopy:

Scanning electron microscopy (SEM) was performed to determine collagen matrix porosity and observe endothelial adhesion to the collagen matrix. After exposure to 78 hour flow protocol, the platforms were fixed in an aldehyde mixture overnight at room temperature followed by fixation with osmium on ice for 4 hours. Post fixation, the platforms were dehydrated in an ascending series of ethanol solutions (50-70-95%) and then critical point dried by CO₂. After drying, platforms were coated with a thin layer of platinum-palladium and high-resolution SEM imaging was performed with Zeiss Supra40 SEM-Electron Microscope.

### Example 5—Liver and Tumor Model

A novel multi tissue-on-a-chip platform was developed to simulate interactions between healthy/tumorigenic liver and breast tumor microenvironments for drug/nanoparticle development and the dynamic transport of fluorescent nanoparticles was assessed in each compartment. The multi tissue-on-a-chip platform consisting of a vascularized breast tumor and healthy liver microenvironments was developed based upon the vascularized platforms described above. To mimic these microenvironments, cell lines of MDA-MB-231 for breast cancer, C3Asub28 for liver cancer, and THLE-3 for healthy liver were used. Microenvironments were fabricated from type I collagen concentrations of 7 mg/ml and 4 mg/ml for tumor and liver respectively to replicate the growth characteristics and compression moduli of these tissues. Fully functional endothelialized vessels within the tumor and liver microenvironments were formed using a graded flow preconditioning protocol. Wall shear stresses of 4 dyn/cm² (healthy) and 1 dyn/cm² (tumor) were employed within each vessel to mimic physiological conditions.

To prove feasibility of microenvironments, cell viability was measured for 3 days and native cell morphology was confirmed with SEM imaging and F-actin/DAPI staining. The fidelity of liver cells cultured in the microenvironment was demonstrated by detecting albumin expression and release in response to physiological shear stress. Dextran particles with sizes of 3 kDa and 70 kDa were per-fused in the platform to replicate the hydrodynamic diameters of chemotherapy drugs and drugs conjugated with nanoparticles. The effect of different co-culture conditions on vessel permeability, ECM/vessel porosity and accumulation of nanoparticles were quantified using intensity profiles in response to different interactions between breast tumor and liver microenvironments to simulate the conditions of drugs being metabolized (liver to breast tumor) and non-metabolized (breast tumor to liver). Ultimately, the physiological multi tissue-on-a-chip platforms developed in this study enabled quantification of drug transport and distribution behavior spatially and temporarily.

The first vascularized multi tissue on-a-chip microenvironments was developed herein for modeling cancerous breast and cancerous/healthy liver microenvironments for studying dynamic and spatial transport of particles. Mechanical properties were tuned to mimic native tissues modeled and cell response, vessel permeability, and porosity of vessel and ECM were assessed. Ultimately, the transport kinetics and accumulation of varying sized fluorescent dextran particles representative of chemotherapeutics and nanoparticle conjugated chemotherapeutics within the tumor and liver microenvironments were determined. The influence of particle delivery to specific tissue microenvironments to simulate direct tumor delivery or metabolism of drugs prior to delivery to the tumor was also investigated.

Cell Morphology and Viability:

MDA-MB-231, THLE-3, and C3Asub28 cell lines were cultured in avascular collagen at concentrations mimicking each tissue's mechanical properties without vasculature for 3 days, and cell morphology for each day was characterized. FIG. 17 shows associated cell morphology using DAPI and F-Actin staining and SEM images. MDA-MB-231 cells developed an elongated, stellate morphology with disorganized nuclei and invasive processes were observed by day 3. Similarly, THLE-3 exhibited an elongated morphology as Pfeifer et al. showed for isolated human hepatocyte cells (Pfeifer et al., PNAS, 1993).

Unlike the elongated healthy liver morphology, liver cancer cells formed clusters and the size of each cluster increased daily as previously shown for polymer based in vitro platforms by Shuler et al. and Li et al. and in vivo samples by Siveen et al (Wang et al., 2006; Siven, 2014). Moreover, SEM images more clearly denote cell morphology and its interaction with the surrounding collagen matrix. C3Asub28 cells possess a rounded shape, contrary to the epithelial THLE-3 morphology and pleomorphic MDA-MB-231 cells with elongated shape. Cell morphology similarities between day 3 and SEM images showed SEM preparation did not affect cell and matrix properties. The observed morphological elongation for healthy liver cells and aggregation behavior of breast and liver cancer cells is due to cell-cell and cell-ECM interaction as previously mentioned by collagen based in vitro studies (Szot et al., 2011).

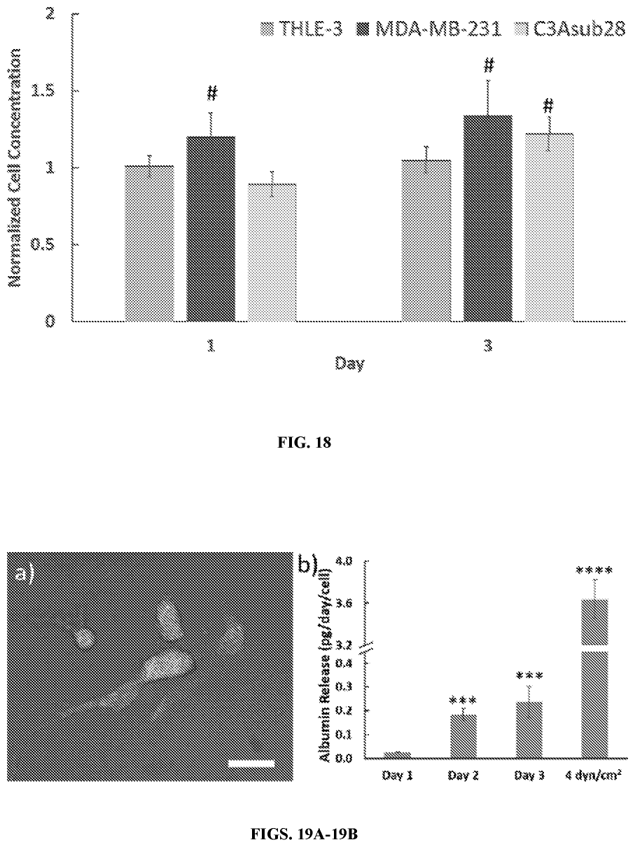

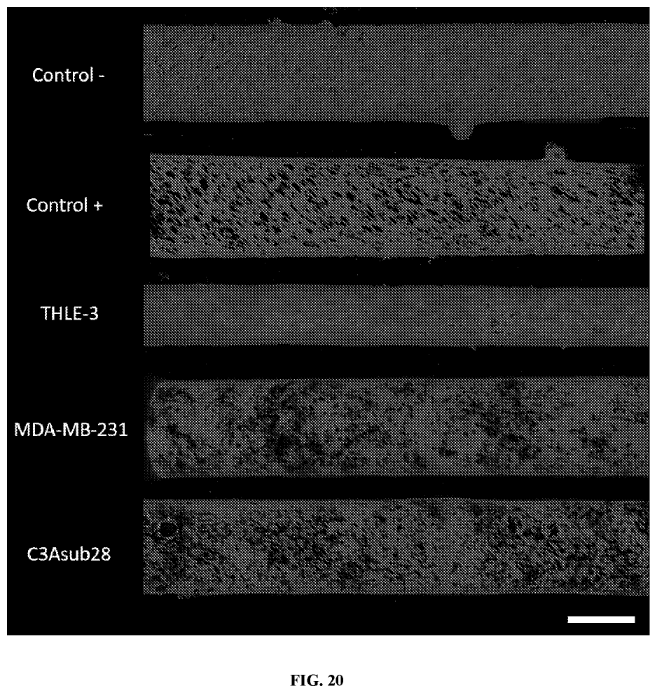

FIG. 18 shows the viability of each cell line over 3 days. As shown, cells were viable over the time course of 3 days within the avascular collagen microenvironments. As expected, cells required some time to adhere before they began to proliferate except for MDA-MB-231 cells which proliferated significantly by 1.20 fold (p<0.05) on the first day. By the third day, C3Asub28 and MDA-MB-231 cells continued to proliferate significantly with 1.23 and 1.34 fold (p<0.05), respectively. Although the THLE-3 concentration did not change significantly, cells remained viable. This data confirmed that the microenvironments supported sustained cell viability and proliferation, which is consistent with our previously published data with MDA-MB-231 vascularized microenvironments (Buchanan et al., 2013).

Albumin Expression and Release of Healthy Liver Cells:

The functionality of healthy liver cells was determined by detecting albumin expression and release. Albumin expression and release was measured for collagen based vascularized THLE-3/TIME microenvironments for the first time in this study. FIG. 19A shows anti-albumin immunostained THLE-3 cells in the collagen microenvironment. Cells exhibited elongated morphology, which is also shown in FIG. 19. The albumin level presented in FIG. 19 shows that the release increased significantly with time compared to day 1 (p<0.005). While the number of cells did not change over the preconditioning period as presented in FIG. 18, the increase of albumin expression can be explained by two main reasons.

First, in FIG. 17 it was observed that cells exhibited a more elongated morphology with time, suggesting cells were becoming more established yielding native genotypic and phenotypic behavior as reported by Szot et al. in which cell morphology and phenotype were characterized within in vitro collagen microenvironments (Szot et al., 2011). Second, increasing wall shear stress with each day promoted greater cellular expression of albumin. Buchanan et al. previously showed that increasing wall shear stress promoted angiogenic gene protein expression of cancer cells cultured in vascularized collagen platforms (Buchanan et al., 2014). A recent study by Maria et al. also demonstrated that albumin release was elevated by increasing the shear stress using in vitro samples (Maria et al., 2011). The albumin level of liver in normal human individuals was reported as 150-250 mg/kg/day and for human biopsy samples it was known that cell concentration is 0.65-1.85×10⁸ cells/g. Using the cell concentration and albumin level reported in these studies, cell-wise human albumin release was calculated as 0.81-3.85 pg/cell/day. The measured albumin in response to physiological flow was 3.64-0.19 pg/cell/day, lied within the range of published values. This verified the functionality of the developed vascularized healthy liver microenvironment under given flow conditions.

Porosity of Vasculature:

After embedding cells in collagen and successfully preconditioning endothelialized channels for 72 hours to establish a confluent, aligned endothelium, the effect of different co-culture conditions on the vessel confluence was studied as shown in FIG. 20. The first two cases involved creation of platforms with only a functional endothelium and no cells within the ECM, referred to as a TIME monoculture (Control −) alone or in the presence of TNFα being perfused in the vessel (Control +) to dilate the vessel pores for comparison with previous permeability result by Zervantonakis et al. (Zervantonakis et al., 2012).

The last three conditions incorporated different cell types within the collagen ECM in addition to the TIME culture: C3Asub28/TIME, MDA-MB-231/TIME, THLE-3/TIME microenvironments. A tight confluent endothelial lumen in which fluorescence is shown with minimal dark gaps between cells is apparent for the Control −. The vascularized endothelium co-cultured with THLE-3 showed a very similar endothelial confluency compared to Control −.

Moreover, it was observed that artificial modulation of the vessel with TNFα treatment, (Control +), caused vessel permeabilization with significant pore openings compared to Control −. On the other hand, the tumor vessels exhibit a patchy and leaky endothelium with perivascular detachment and non-uniform gaps unlike the uniform, dilated openings of Control +. This strengthened the idea that the cross-talk between cancer and endothelial cells cause a leaky porous domain, leading to the well-known EPR effect, also shown in vascularized tumor microenvironments₃₃. Vessel porosity of varying vascularized tissue microenvironments was reported for the first time in this study and presented in FIG. 14A. Based on the measured results, inclusion of breast and liver tumor cell lines in the platform increased vessel porosity by 2.64 (p<0.001) and 3.62 fold (p<0.001) respectively compared to the Control −. This was an evident phenomena that the cross-talk and signaling between cancer and endothelial cells and release of TNFα caused detachment of endothelial cells that created large gaps around the vessel surface.

It was observed that co-culture with THLE-3 did not affect vessel porosity significantly compared to the Control −. Therefore, the change observed for endothelial integrity in cancer microenvironments compared to Control − was mostly likely due to signals provided by the cancer cells. One other reason for the observed patchy endothelial structure could be due to the heterogeneous distribution of cell clumps observed in FIG. 17 liver and breast cancer cells located in the ECM. Cell aggregation leads to non-uniform release of expressed proteins across the ECM and vessel resulting in cells invasion into the endothelial layer over time as presented by collagen based vascularized microenvironments. The amount of released protein perfusing through the leakier endothelial layer of the liver cancer microenvironment was expected to be greater due to 1.37 fold higher porosity (p<0.05) compared to breast cancer.

Ecm Porosity:

Following transport through the endothelium, nanoparticles or drugs must navigate the ECM to reach the tumor cell, therefore, the ECM structural properties of the microenvironments were characterized. Collagen concentrations of 7 mg/ml and 4 mg/ml were used to create the ECM to replicate the compression modulus of tumor and healthy liver microenvironments. SEM images presented in FIG. 14B show fiber alignment was caused by shear stress during preconditioning, which is different than randomly oriented static ECM images.

Quantified ECM porosity results presented in FIG. 14C showed that ECM porosity in MDA-MB-231 microenvironments did not change significantly but C3Asub28 microenvironments increased by 1.14 fold (p<0.05) relative to the vessel only Control −. Schedin et al. also previously indicated that in vitro mechanosignaling events carried out by cancer cells can alter ECM stiffness and consequently porosity (Schedin and Keely, 2011). Furthermore, THLE-3 microenvironment ECM porosity increased by 1.36 fold (p<0.005) compared to higher collagen concentration of Control −. As the type of cell line embedded in the collagen affects vessel and ECM porosity, the ability to transport of drug is also expected to be altered correspondingly.

Vessel Permeability of Microenvironments:

Permeability was measured using for two different dextran particle sizes (3 and 70 kDa) for 5 different types of microenvironments (acellular (no endothelial cells lining the vessel and no cells in the ECM), TIME monoculture (endothelialized vessel with no cells in the matrix), C3Asub28/TIME, MDAMB-231/TIME and THLE-3/TIME microenvironments). The permeabilities of C3Asub28/TIME and THLE-3/TIME microenvironments were measured for the first time in this study.

Accordingly, permeability of mentioned microenvironments are presented in FIG. 14D, which provide a measure of the leakiness of the endothelial lumen for each given condition as a function of particle size. The results showed higher permeability for acellular (cell free) microenvironments for both particle sizes since the lack of an endothelial barrier does not regulate transport. There was a significant decrease in permeability when THLE-3 cells were cultured in the ECM, despite having higher ECM porosity. This is due to having higher shear stress, which gives less time for particles to diffuse through the ECM.

However, the presence of cancer cells such as MDA-MB-231 and C3Asub28 with endothelial cells caused higher permeability compared to endothelium monoculture and THLE-3/endothelial microenvironment. The difference between normal and hepatocellular carcinoma was observed as evidenced by cancerous cells increasing permeability by 2.77 (p<0.001) and 2.35 (p<0.05) fold for 70 and 3 kDa particles, respectively. Previous studies on vascularized tumor-endothelial microenvironments also showed similar findings in which an increase in transport of macromolecules occurred due to inclusion of cancer cells (Jain et al., 2014).

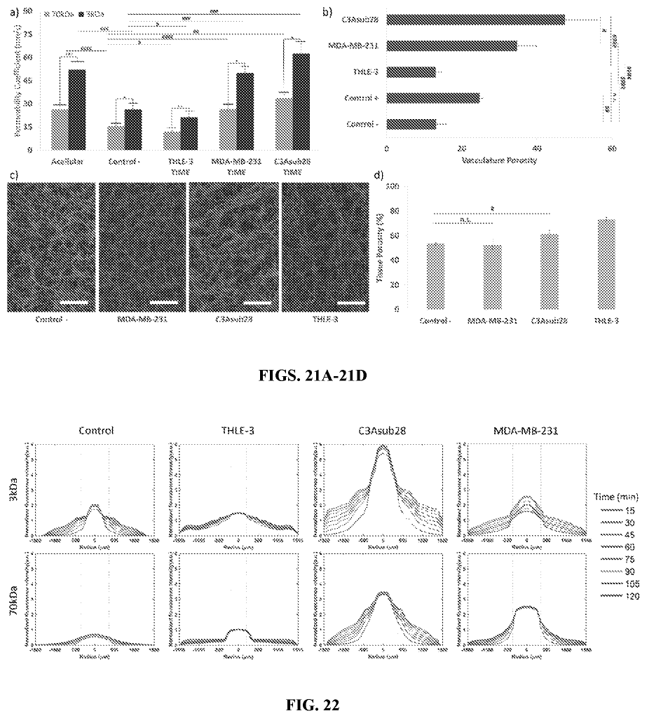

There are two underlying reasons for this difference between the two liver cell lines. First, drug has been perfused through normal liver with higher wall shear stress to generate physiological transport. Secondly, due to interaction between cancer and endothelial cells or tumorigenic protein release by cancer cells, endothelial layer porosity decreases, which has been discussed in the previous section. This phenomenon is described as the EPR effect and is more significant compared to high wall shear stress. The presence of tumor cells inside the ECM increased vasculature permeability increasing the likelihood of tumor cell invasion and migration into the endothelial layer. Cancer cells significantly influenced the endothelium as evident by large pores shown in FIG. 20. This more porous endothelial layer caused higher permeability as presented in FIG. 21. The vasculature permeability and porosity are indicative of the transport properties, but the impact of particle size on vessel regulation is a key determination in particle delivery and accumulation.

For all microenvironments, 3 kDa dextran particle were more permeable in the microenvironments than 70 kDa. In vivo drug testing studies have shown that nanoparticle size highly influences permeability (Venkatasubramanian et al., 2008). The relationship between hydrodynamic diameter and permeability coefficient can be explained using Stokes-Einstein Equation of diffusivity (Yuan et al., 1995). By definition, particle size is indirectly proportional to permeability, which is indicated with higher diffusivity of smaller particles. Therefore, more rapid diffusion was observed for 3 kDa particles compared to 70 kDa which results in a higher permeability coefficient. Moreover, the presence of endothelial layer around the vasculature yielded a reduction in permeability coefficient of dextran particle since matrix pore openings were blocked with endothelial monolayer as described in FIG. 20.

The validity of permeability measurements were assessed by comparing the fold change between the same Control − and Control + findings in the literature. Collagen based vascularized breast cancer platform developed by Zervantonakis et al. reported the fold change between Control − and Control + as 1.79±0.27, while is similar to our measured value of 1.59±0.13 in this study. Moreover, permeability coefficient of 70 kDa dextran particle was reported as 25.67±1.79 nm/s in vascularized collagen based tumor microenvironments for the same shear stress which compares well with our permeability results (26.83±2.19 nm/s).

Intensity Profiles and Accumulation:

In addition to permeability, intensity profiles of particle fluorescence within the vessel and ECM provided insight regarding the accumulation of each type of particle in the different tissue microenvironments. Spatial and temporal quantification of particle accumulation rate in the ECM and vessel were presented for the first time. 3 and 70 kDa dextran particles were used to mimic chemotherapy and chemotherapy/nanoparticle conjugated drug sizes, specifically for 1.9 and 12.6 nm hydrodynamic diameter, respectively.

FIG. 22 presents intensity profiles for two different particle sizes and four different microenvironments (Control −, C3Asub28/TIME, MDA-MB-231/TIME, and THLE-3/TIME microenvironments). For all these microenvironments, a sharp change was observed in the slope between the vessel and the ECM interface. This is due to the presence of the endothelium which acts as a barrier to particle transport. However, this decay significantly changed for different particle sizes. The intensity rate over time for small particle sizes was more rapid compared to large particles. This trend was observed for all microenvironments simply due to the fact that smaller particles were able to penetrate faster through endothelial pores compared to larger particles.

Additionally, the increase in maximum intensity over time with smaller particle size at the center of the vessel was observed for tumor cell lines. For microenvironments containing tumor cells, peak intensity of small particle was normalized to large particle sizes and found to be 1.66 and 1.59 fold for MDA-MB-231 and C3Asub28 microenvironments, respectively. This trend can be explained by both collagen based vascularized 3D in vitro tumor microenvironments and modeling studies (Buchanan et al., 2014) for two main reasons. i) Advective transport through the vessel is more dominant than diffusive Brownian motion into the ECM. ii) Particles are diffusing and then leaving the ECM which causes accumulation around the vessel. Given the fact that vessel porosity in tumor microenvironments is significantly higher compared to healthy tissue microenvironments as seen in FIG. 21, particles can rapidly penetrate into the ECM. Furthermore, particles are able to freely diffuse back from the ECM to the vessel since the leaky endothelial layer fails to trap particles inside the ECM. The magnitude of the liver carcinoma intensity profile is much higher than all other microenvironments.

Moreover, the peak intensity for liver tumor microenvironments increased by 2.77 and 4.48 fold (p<0.01) for 3 and 70 kDa, respectively. A similar trend was observed for breast tumor microenvironments in which 1.39 and 3.65 fold (p<0.05) increases occurred for 3 and 70 kDa respectively, whereas healthy liver peak intensity did not change significantly compared to Control −.

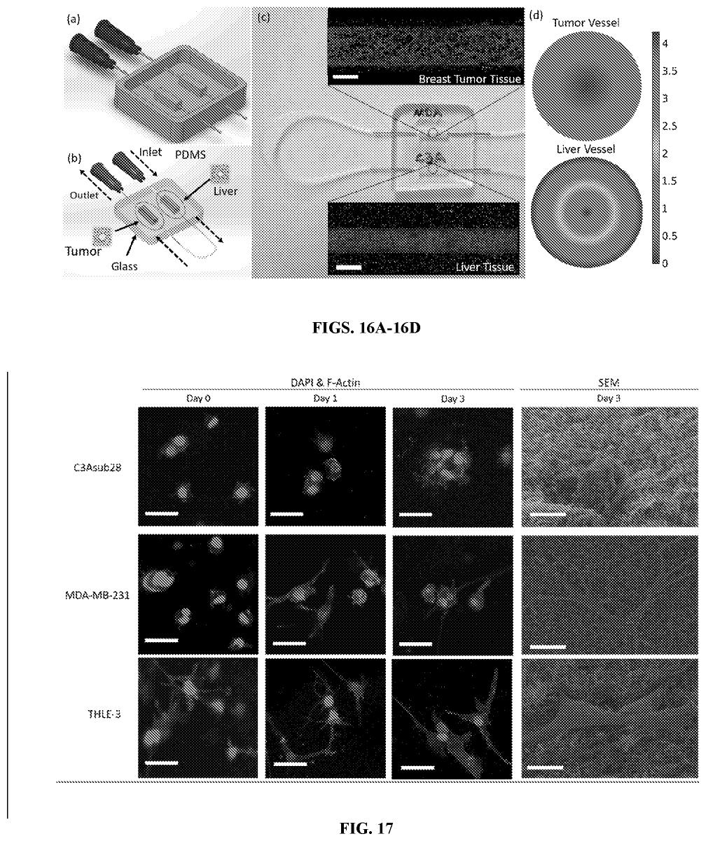

Connecting the vascularized liver and breast tumor microenvironments in series and perfusing particles in either vessel enabled simulation of the accumulation behavior of metabolization of particles (liver to tumor) or direct delivery to the tumor (tumor to liver). With both microenvironments connected, independent of which microenvironment received particles first, a significant decrease was observed in the magnitude of the intensity in the second microenvironment (FIG. 16) for circulation in two microenvironments compared to perfusion through a single microenvironment alone (FIG. 17) expected that the first microenvironment retains some portion of supplied particles. Moreover, the peak intensity value for MDA-MB-231/TIME microenvironments after passing through THLE-3/TIME microenvironments decreased by 2.40 and 1.99 fold (p<0.05) for 3 and 70 kDa respectively, compared to circulation of particle in the MDA-MB-231/TIME microenvironment alone. This showed that particles had already been uptaken by healthy liver cells which simulates the drug being metabolized by the liver, which eventually causes liver injury or failure for many chemotherapeutics.

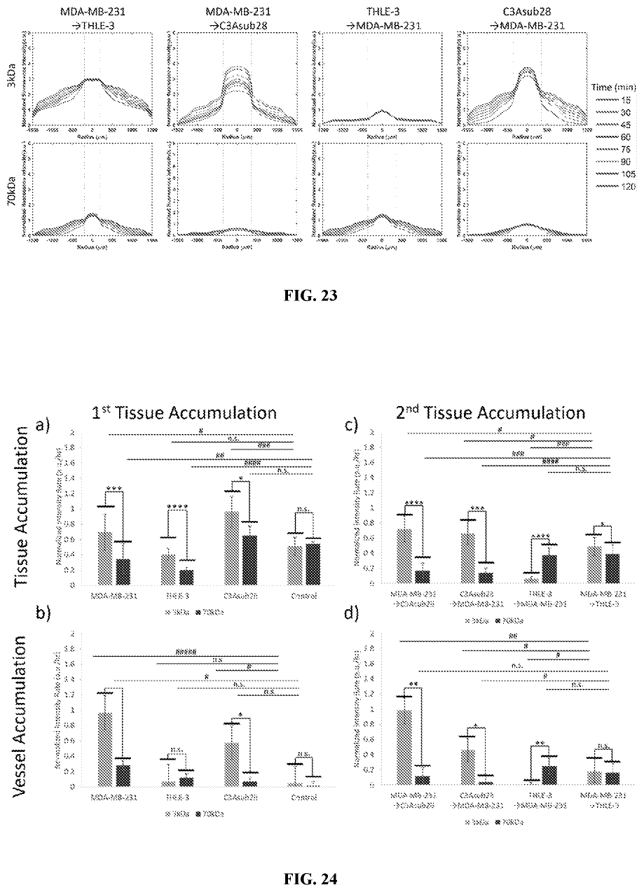

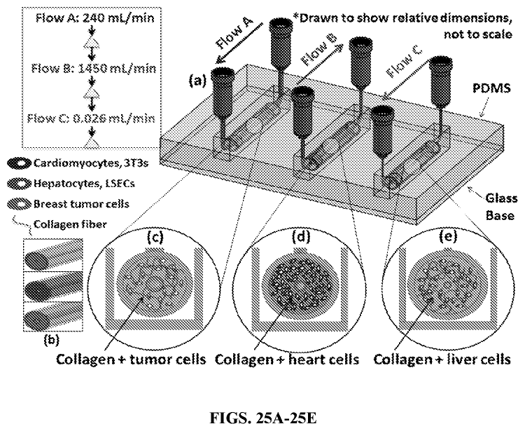

However, as healthy liver was replaced with liver tumor, breast tumor peak intensity increased by 1.31-fold (p<0.05) for 3 kDa and decreased by 2.60 fold (p<0.05) for 70 kDa. To gain further understanding, the intensity rates were determined using data from FIGS. 22 and 23. Calculated accumulation resulted in the vessel and ECM presented in FIG. 24. Intensity rate presented in FIG. 24A showed particle accumulation in the ECM in carcinoma microenvironments are significantly higher than the healthy liver microenvironment.

This phenomena can be explained with two main reasons: First, the leakiness of endothelial layer causes EPR effect and second, significantly lower shear stress allows more time for particles to diffuse through the ECM similar to work described by Buchanan et al. regarding permeability change with respect to wall shear stress. Although it was concluded that ECM porosity of THLE-3/TIME microenvironment is higher than the ECM of both cancer microenvironments, particle accumulation in the MDA-MB-231/TIME and C3Asub28/TIME microenvironments are 3.45 and 4.81 fold (p<0.05) higher than healthy liver, respectively. This indicates that vessel porosity plays a more dominant role compared to ECM porosity in the accumulation rates of particles in ECM. Particle accumulation in the vessel on the other hand is a factor which should be controlled since this will enhance the likelihood of particles being delivered to other healthy tissues causing toxicity as is shown by in vivo drug distribution studies (Dong et al., 2015).

Also, having higher ECM accumulation in the liver cancer compared to breast cancer was anticipated based on having higher ECM porosity presented in FIG. 21D. Particle accumulation in vessel presented in FIG. 24B shows the ECM of MDA-MB-231/TIME and C3Asub28/TIME microenvironment have 3.45 (p<0.05) and 8.11 (p<0.01) fold higher accumulation for small particle sizes compared to large particle sizes. However, the vessel accumulation for large particle size was not significant except for MDA-MB-231/TIME microenvironments with 17.67-fold change (p<0.05). This is due to particles passing the leaky endothelial barrier and diffusing back to the vessel, which was not observed for the healthy liver microenvironment because of the tight endothelial lumen.

When multiple microenvironments were connected however, particle accumulation was expected to change due to particles remaining in the microenvironment perfused first before entering the second microenvironment. FIG. 24C presents the accumulation of particles in the ECM for varying circulation patterns between microenvironments. An interesting result was observed for ECM accumulation for THLE-3/TIME to MDA-MB-231/TIME and MDA-MB-231/TIME to THLE-3/TIME microenvironments. ECM uptake of MDA-MB-231/TIME decreased by 6.98-fold (p<0.01) after passing the THLE-3/TIME microenvironment and THLE-3/TIME ECM accumulation increased by 2.46 fold (p<0.05) after particles passed through the MDA-MB-231/TIME for smaller particle size which represent chemotherapeutics. This result presented the significant disadvantage of metabolized and non-metabolized (direct delivery to tumor) cases for localization of particles in the tumor. On the other hand, large particles, which represent chemotherapy-nanoparticle conjugation, decreased THLE-3/TIME ECM accumulation by 2.57 fold (p<0.01) and increased MDA-MB-231/TIME microenvironment accumulation by 5.57 fold (p<0.01) compared to small nanoparticles.

In vivo studies with similar particle sizes also emphasized that nanoparticles with hydrodynamic diameter close to 15 nm have greater probability of accumulation in the tumor. Moreover, vessel accumulation under the interaction significantly decreased in secondary microenvironment in all cases as seen in FIG. 24D. Based on these results, using chemotherapy alone may be less advantageous compared to chemotherapy nanoparticle conjugation within the size range tested in this study.

This outcome was observed for both cases in which the particles being metabolized (liver to tumor) were simulated as being directly delivered to the tumor (tumor to liver). When small particles were perfused through the liver first, breast tumor ECM accumulation was decreased by 5.49 fold (p<0.01) compared to perfusing through the tumor first. Simulated metabolized and non-metabolized cases where healthy liver was replaced with liver tumor cells, particle accumulation was decreased by 1.05 and 3.94 fold (p<0.05) for 3 and 70 kDa particle sizes, respectively.

Thus, it was shown that the multi tissue-on-a-chip devices has greater potential than standard cell culture, static in vitro setups, and if the system is complex enough, it can augment or replace animal testing for advanced drug development before clinical studies. The tissue on-a-chip microenvironment developed in this study provides a system that mimics transport in vivo enabling spatial and dynamic assessment of transport of any type of drug/nanoparticle as a function of their size. This device can be used to investigate the influence of other drug/nanoparticle properties including surface charge, dimensionality, targeting ligand, and aspect ratio on transport. By altering the direction of flow the effect of targeting and metabolism on transport kinetics of drugs/chemicals can be simulated in high throughput, inexpensive optimization of nanoparticles or other therapeutics by enabling toxicity, efficacy, and biodistribution measurements as a function of varying microenvironmental conditions and drug/nanoparticle properties. The multi tissue-on-a chip microenvironments can also be utilized for testing a combination of different treatment methods such as hyperthermia, radiation, and a myriad of nanoparticles with unique functionality to create solutions for targeted delivery.

### Example 6—Materials and Methods

Human Cell Sources: Human breast cancer cells (MDA-MB-231), healthy liver cells (THLE-3), carcinoma liver cells (C3Asub28), and telomerase immortalized microvascular endothelial cells (TIME) were used in this study. MDA-MB-231 cells (American Type Cell Culture, ATCC, VA, HTB-26) were cultured with Dulbecco's Modified Eagle's medium, nutrient mixture DMEM/F12 (1:1)+LGlutamine, +15 mM HEPES (Invitrogen, CA) supplemented with 10% fetal bovine serum (FBS, Sigma Aldrich, MO), and 1% Penicillin/Streptomycin (P/S, Invitrogen, CA). TIME cells stably transduced with an mKate lentivirus were generously provided by the Wake Forest Institute of Regenerative Medicine, Winston-Salem, N.C. These cells were cultured in Endothelia Basal Medium-2 (EBM-2, Lonza, MD) and supplemented with an Endothelial Growth Media-2 (EGM-2) SingleQuotsO Kit (Lonza, MD), which contains 2% FBS, hydrocortisone, Vascular Endothelial Growth Factor (VEGF-2 ng/mL), Human Fibroblast Growth Factor-basic, (hFGF-B, 4 ng/mL), R3-insulin growth factor, ascorbic acid, human epidermal growth factor, GA-1000, and heparin. Human carcinoma liver, C3Asub28 cells were generously provided by Dr Wei Li from the University of Texas at Austin. These cells were cultured with DMEM/F12 (1:1)+L-Glutamine, +15 mM HEPES with 10% FBS, and 1% P/S. THLE-3 cells (ATCC, VA, CRL-11233) were cultured in BEGM Bullet Kit (Lonza, MD) with additional 5 ng/mL Epiderman Growth Factor (EGF, Invitrogen, CA), 70 ng/mL Phosphoethanolamine (Acros Organics, Belgium), and 10% FBS in a pre-coated flask. All cells were incubated at 37° C. and 95% atmospheric air/5% CO₂. Cell growth was monitored every day and cells were used in experiments when they were 70% confluent. All cell lines were used at the first 8 passages.

Tissue Properties and Preparation of Collagen:

Type I collagen was used as the primary extracellular matrix (ECM) component for each tissue microenvironment. Stock solution of type I collagen was prepared by dissolving excised rat tails in an HCl solution at a pH of 2.0 for 12 hours at 23° C. (Buchanan et al., 2013). The solution was then centrifuged at 23° C. for 45 minutes at 30000 g and supernatant was collected and lyophilized. The lyophilized collagen was mixed with diluted 0.1% glacial acetic acid, maintained at 4° C. and mixed every 24 hours for 3 days to create a collagen stock solution. Finally, collagen was centrifuged at 4° C. for 10 minutes at 2700 rpm to remove air bubbles. Since ECM stiffness directly affects cell-matrix interactions, such as cell adhesion/proliferation, and diffusivity of drugs into the tissue, and that collagen concentration dictates ECM mechanical properties, it is critical to select an appropriate final collagen concentration to mimic human desired tissue properties, which also controls tissue porosity. Yeh et al. reported that hepatic tumor microenvironment has stiffness of 3 kPa (Yeh et al., 2002). Similarly, breast cancer tissue stiffness is reported as 4 kPa. Chen et al. reported the healthy human liver compression modulus varies between 0.59-1.73 kPa (Chen et al., 1996). Therefore, the final collagen concentrations for liver and breast carcinomas of 7 mg/ml were employed since previous studies reported corresponding compression modulus of 3-6 kPa (Buchanan et al., 2013). A collagen concentration of 4 mg/ml was used to create the normal liver tissue with corresponding compression modulus of 0.90-1.91 kPa.

Device Design and Fabrication:

An aluminum mold, was fabricated using micromilling techniques, which eliminates multistep fabrication processes and necessity of expensive patterning reagents compared to conventional fabrication technique (photolithography). Well mixed Polydimethylsiloxane (PDMS) with curing agent of 10:1 ratio was poured inside the aluminum mold and baked for 1 hour at 75° C. Solidified PDMS, which is the housing material, consists of inlet and outlet channels, was peeled off from the mold and sterilized under UV for 1 hour with 25×25 glass slide before the bonding process. Then, glass slide and PDMS were plasma treated (Harrick Plasma) and bonded to create the enclosure to surround the tissue microenvironment. To increase adhesion between collagen and PDMS housing, fabricated PDMS housing assembled with glass slide was filled with sterile 1% Polyethylenimine (PEI, Sigma-Aldrich, MO) (diluted with DI H₂O) and incubated for 10 minutes. After aspirating PEI, channels were filled with 0.1% glutaraldehyde (Sigma-Aldrich, MO) (diluted with DI H₂O) and incubated for another 20 minutes. Glutaraldehyde was removed and the platform was washed twice with sterile DI H₂O. Collagen solution was neutralized to pH of 7.4 with 1×DMEM, 10×DMEM and 1N NaOH and mixed with intended cell line at a concentration of 1×10⁶ cells/ml. Collagen-cell mixture was injected into the platform to fill the enclosure. Final concentrations of collagen was selected as 4 and 7 mg/ml for healthy and tumorigenic tissues, respectively, to match human compression modulus of relevant tissue type. A needle was inserted inside the platform to form hollow vessel before the polymerization of collagen. The needle size of 22 and 27G (Jensen Global, CA) were used for tumor and healthy liver tissues, respectively, to provide the relevant physiological wall shear stress (WSS) in the tissues. Applied wall shear stress is a significant phenomenon to mimic human tissue as well as protein release by the cell lines. In a clinical study performed by Karin et al. showed that human wall shear stress in vessel varies between 1-10 dyn/cm² as well as wall shear stress decreases down to 1 dyn/cm² for tumor microenvironments. However, wall shear stress higher than 4 dyn/cm² showed decrease in albumin release according to in vivo study. Therefore, needles at given size were inserted respectively for tumor and healthy liver microenvironments to provide 1 and 4 dyn/cm² wall shear stresses at the same flow rate. After the incubating the platform for 30 minutes at 37° C. and 5% CO₂, the collagen was polymerized and presence of needles created a hollow vessel inside housings.

To create a fully functional aligned endothelium along each channel within each compartment, TIME cell suspension in media (10×10⁶ cells/ml) was introduced in the channel and underwent flow preconditioning for 3 days. Within the first 36 hours, wall shear stress was maintained at 0.01 dyn/cm² and followed with a linear increase of wall shear stress to 0.1 dyn/cm² for 1 hour and maintained at this value for the next 36 hours. In the last 6 hours, wall shear stress was linearly increased to physiological wall shear stress. For positive control samples, 20 ng/ml Tumor Necrosis Factor Alpha (TNFα, RnD Systems, MN) was perfused at 0.1 dyn/cm² for 24 hours after the preconditioning protocol before transport studies. To provide flow into the microfluidic platform, 0.5″ long 22G stainless steel needles were inserted though PDMS ports and partially into the collagen microchannels. Autoclaved Tygon silicon tubing ( 1/16″ ID, Saint Global, France) was connected to the inlet needle and a bubble trap, which is connected to a syringe pump that controls the flow rate. The bubble trap eliminates the likelihood of washing out endothelial cells from the created vessel with the effect of introduced bubble in the platform channel. The outlet needle was similarly connected to silicon tubing that collects the media into a reservoir. Two chambers were connected using 22G pins and the same silicon tubing. Detailed images of the platform before and after assembly and preconditioning are given in FIG. 16.

The viability was assessed in avascular platforms to measure growth kinetics of cells located in the ECM. Identical platform preparation protocol was followed as described in the previous section without incorporation of an endothelialized channel. To maintain consistency avascular platforms were cultured with endothelial cell culture media to maintain cell viability. Cell viability was measured using CellTiter-Blue (Promega, WI) Assay over the course of three days. Fluorescent intensity units was converted to cell concentration using the obtained calibration data in this work.

Cell Morphology:

Cell morphology at day 0, 1, and 3 were determined as described previously. Briefly, avascular platforms were fixed with 3.7% paraformaldehyde and permeabilized using 0.1% Trition X-100 (Sigma Aldrich, MO). Then, samples were blocked with 1% BSA (Santa Cruz Biotechnology Inc, CA) for 30 minutes at room temperature followed by an incubation step with rhodamine phalloidin (Invitrogen, CA), a high-affinity probe for F-actin. Samples were counterstained with DAPI (Vector Laboratories, CA), to visualize nuclei. Imaging was performed using Leica SP8 laser scanning confocal microscope. Another set of vascularized platforms were fixed to investigate cell morphology and ECM porosity using Scanning Electron Microscopy (SEM). Aldehyde mixture composed of 0.2 M cacodylate buffer, glutaraldehyde, paraformaldehyde, cation stock, and DI H₂O were prepared and fixed at room temperature for 4 hours and washed three times with cacodylate buffer for 15 minutes. Subsequently, reduced osmium solution (1:1) composed of 4% potassium ferrocyanide in 0.2 M cacodylate buffer and 4% aqueous osmium tetroxide was added to samples and maintained on ice for 4 hours. Fixed samples were washed with DI H₂O 5 times for 10 minutes afterwards and dehydrated with 50, 70, and 95% ethanol once and twice with 100% ethanol for 15 minutes each. Samples were dried using a critical point drying method, coated with 12 mm thick Pt/Pd layer and imaged with Zeiss Supra40 SEM. All reagents were purchased though Electron Microscopy Sciences, PA.

Assessment of Transport Properties and Quantification:

Transport measurements of varying particle sizes in the multi tissue-on-a-chip microenvironment were conducted for two different scenarios: single microenvironment analysis and microenvironments connected in series to investigate the influence of their interactions and interdependent transport kinetics. When considering only a single microenvironment transport, particles were delivered through the vessel of the microenvironment of interest and transport through the vessel and into the surrounding ECM was measured subsequently and spatially. In these tests, 5 different platform configurations was used: acellular with no cells in the ECM and no cells in the vessel, TIME monoculture consisting only endothelial cells lining the vessel without cells in the ECM, and then vascularized microenvironments denoted as cells in the ECM/cells in the vessel: MDA-MB-231/TIME, C3Asub28/TIME, and THLE-3/TIME microenvironments. Microenvironments were connected in series to consider the influence of interactions between them. Particles were perfused through the first microenvironment's channel with associated diffusion into the corresponding ECM and back into the vessel which resulted in transport to the next tissue compartment. Four different multi tissue-on-a-chip configurations were considered: MDA-MB-231 to C3Asub28, MDA-MB-231 to THLE-3, THLE-3 to MDA-MB-231 and C3Asub28 to MDA-MB-231. Cases when particles were introduced directly in the vessel corresponding to the breast tumor was meant to simulate direct delivery to the breast tumor, where particles are not metabolized, and cases when particles were first introduced into the vessel associated with the liver simulated metabolization by the liver. Passive transport of particles through blood vessels within the previously described microenvironments depends on the permeability of the each vessel endothelialium and the porosity of the vessel and ECM of each tissue. Particle transport begins in the blood vessel, which is surrounded by endothelial cells and ECM. Endothelial integrity controls the barrier function and regulates transport of particles. According to in vivo studies, the gaps between endothelial cells are significantly higher in tumors vessels compared to healthy tissue vessels, and is referred to as the enhanced permeability and retention (EPR) effect. Furthermore, this leakiness of the endothelium may also allow particles to diffuse back into the vessel from the ECM, which creates the vessel accumulation. Additionally, the ECM can act as a sink trapping particles leading to accumulation within the tissue. Therefore, ECM and vessel porosity and permeability, which affect intravasation and extravasation of particles need to be characterized to fully describe expected transport of particles. The effect of porosities on diffusion is also stated by Darcy's Law given in Equation 7: u=K∇P/ζμ=z m (7) where μ is velocity in the porous domain, m is viscosity, ∇P is the pressure gradient vector, and K is hydraulic permeability. This equation suggests porosity (ζ) within the vessel and ECM determines the effectiveness of particle transport through ECMs. The velocity in the porous domain depends on porosities in each domain which will consequently affect permeability and transport of the vessel and ECM.

Therefore, endothelial porosity was determined using fluorescence microscopy images of mKate tagged endothelial cells. ECM porosity is obtained by analyzing SEM images of ECMs as described in the previous section using ImageJ. Selection of particle size is an important factor that controls the circulation time, tumor uptake, and ability of the particle to penetrate the tissue. Common chemotherapy drugs used for breast cancer treatment such as doxorubicin has hydrodynamic diameter in the range of 1.06-1.89 nm, which can also calculated using molecular weight and density of drug. Although the hydrodynamic diameter of nanoparticle chemotherapy conjugated drugs has great variability depending on nanoparticles type, size, and shape, it has been shown that common nanoparticle-chemotherapy conjugated drug size varies between 5-50 nm. In this study, 3 and 70 kDa dextran particle sizes (Sigma-Aldrich, MO), with hydrodynamic diameter of 1.9 nm and 12.6 nm respectively, were selected representing chemotherapy and chemotherapy-nanoparticle conjugated drugs, respectively, to demonstrate the EPR effect on the developed microenvironments. The effect of vessel and ECM porosities and particle size on transport were quantified using two methods, permeability coefficient and intensity profiles of the particles in the vessel and ECM. Fluorescent dextran particles suspended in serum free (to prevent nanoparticle aggregation) endothelial basal media (EBM-2) to the final concentration of 10 mg/ml were perfused through the vascularized microenvironment for 2 hours with a flow rate of 260 mL/min, which yields physiologically representative shear stress in both microenvironments considered with appropriate vessel diameter. Images were taken every 3 minutes using a Leica SP8 Confocal Microscope. Obtained images were exported to Matlab to quantify intensity readings at each time step. For the first method, permeability coefficient was calculated using Equation 8

$${P}_{d} = \frac{1}{{I}_{1} - {I}_{b}} ( \frac{{I}_{2} - {I}_{1}}{Δ t} ) \frac{d}{4} \qquad ( 8 )$$

where I_{b} is the background intensity, I₁ is the average initial intensity, I₂ is the average intensity after recovery, time interval Δt(s), and d(μm) is the diameter of the microchannel. By definition, this parameter quantifies the ability of particles to penetrate from the microchannel to vessel wall then to the ECM and allows observation of the EPR effect. The last five or more consecutive data points from the 2 hours of flow were used to calculate Pd. Data was expressed as a mean value ±standard deviation. TNFα stimulation of endothelialized vessels within the microenvironments modulates permeability, which has been shown by Zervantonakis et al., Accordingly, TIME monoculture (Control −) and TNFα treated endothelial vasculature monoculture (Control +) were prepared according to previously published permeability results and fold changes were compared to these studies validating the accuracy of experiments. For the second method, transport was quantified based on intensity profiles across the ECM boundaries when only one microenvironment was considered or when two microenvironments were connected in series with one another. Additionally, the same data was used to quantify the intensity change in the vessel and ECM to observe the rate of accumulation of different particle sizes in each compartment.

Statistical Analysis:

Student's t-test was used to identify the significance level of differences between multiple data sets. In all subsequent figures, p-values are denoted with either asterisks (*) or pound symbols (#) as follows: (*) or pound symbols (#) as follows: * or #: significant at p<0.05, ** or ##: significant at p<0.01, *** or ###: significant at p<0.005, **** or ####: significant at p<0.001. The number of replicates varied depending on the test type: N=4 for viability test, N=5 for ELISA measurements, N=3 for permeability coefficient and accumulation analysis, N=4 for ECM porosity and images were taken at 4 different locations of the ECM, and N=4 for vessel porosity.

### Example 7—Multi-Organ System

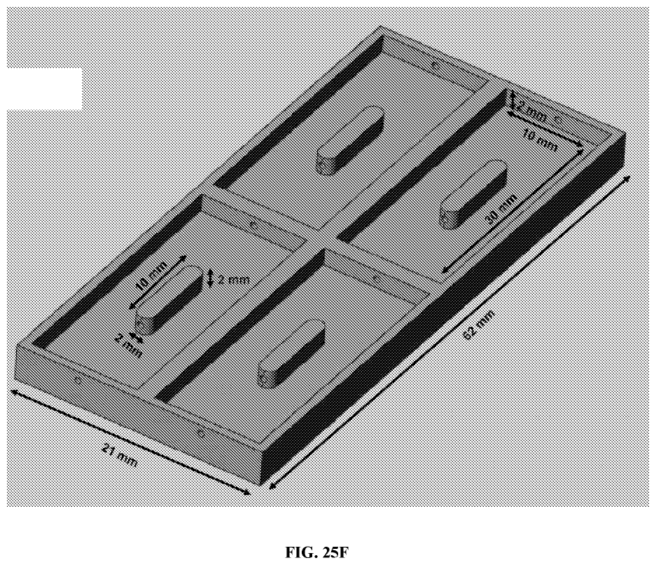

FIG. 25 illustrates the proposed multi-organ system containing vascularized tumor, liver, and heart. Each vascularized tissue module is created using a subtractive needle method (Chrobak et al., 2006). These modules are connected to 90° dispensing needles before being placed in platform chambers. It was shown the capability to scale tissues and vessels, and to produce smaller tissue modules or vessels (<100 microns) with new additive vascular and tissue patterning methods (Landau and Lishitz, 1987). The platform housing consists of a bottom layer made of glass for imaging and the top layer will be fabricated by casting polydimethylsiloxane (PDMS) against a machined aluminum mold to provide slots for holding the interchangeable vascularized tissue modules in place. The two layers can be bonded using plasma treatment. The vascularized tissue will be connected with tubing in series or parallel depending on the purpose of testing. The multi-organ perfusion system is operated using a medium circulation system driven by multiple syringe pumps, each drawing from a reservoir of culture media to compensate for varying flow rates and media consumption by the different tissues. The plug-and-play connection enables interaction of all microenvironments to be explored by altering micro-tubing connection. To consider the influence of all microenvironments, compartments are connected in a physiological order so circulation will flow from liver (if metabolized) to tumor and heart, elucidating the influence of liver metabolism and cardiac toxicity for dose schedules. Changing interconnectivity and order of the microenvironments can broaden the analysis.

Matrix Properties:

The extracellular matrix (ECM) and cellular composition, mechanical, and diffusion properties of the in vitro platform, will be compared to and tuned to match in vivo and patient data for breast tumors, liver, and heart. Collagen concentration influences diffusivity, porosity, and stiffness of the hydrogel, which affects NP-cell-matrix interactions, including matrix remodeling and NP diffusion. Collagen type I will be isolated from rat tail tendons and reconstituted to concentrations of 6-12 mg/mL to form the extracellular matrix for all tissues. This concentration range will yield elastic moduli representative of breast tumors while also maintaining integrity of microchannels under flow and promote cell proliferation. Collagen concentration will be tuned to match Young's modulus for in vivo myocardium (20 kPa-500 kPa) and liver (300 Pa-600 Pa) and verified by measuring the elastic modulus using confined uniaxial compression as we have published.

Cell Culture and Characterization:

The matrix and cellular composition will be tuned to match histology data from respective in vivo tissues. A transformed human microvascular endothelial cell line tagged with mCherry (TIME) will be introduced to form the endothelialized microchannels of all tissues. For the tumor, MDA-MB-231 and inflammatory breast cancer cells (IBC) are labeled with green fluorescent protein, in separate experiments. For the liver, primary hepatocytes and liver sinusoidal endothelial cells or the HepG2 derivative C3A-sub28 cell line with enhanced expression of CYP3A4 mRNA and CYP3A4-mediated activity are cultured in collagen. For the heart, cardiomyocytes (ATCC PCS120010) and 3T3 fibroblasts (ATCC) are used. Prior to inclusion in collagen, hepatocytes, cardiomyocytes, and C3A-sub28 cell lines are stably transfected to express GFP, enabling visualization of cells with confocal laser scanning microscope. All cells (1×10⁶-100×10⁶ cells/ml) are suspended in collagen during polymerization. TIME cells are injected in the microchannel at a density of (10×10⁶ cells/ml), a quantity sufficient to form a confluent lumen. FIG. 25 shows flow rate through each vessel. The system is housed in an incubator while culturing cells in the platform and forming a confluent endothelium. A 3-day graded increase in flow rate preconditioning protocol developed and published by Rylander to create a confluent aligned endothelium for all tissues (Buchanan; incorporated herein by reference). After 72h preconditioning wall shear stresses of T=1, 4, or 10 dyn/cm² is used. Measurement of dynamic spatial and temporal imaging of drug/NP transport/biodistribution, toxicity, and efficacy in the platform (aims 2 and 3) occurs in a live cell incubation chamber coupled with our laser scanning confocal microscope. Prior to imaging, drugs/NPs are introduced in the media for circulation through tissue chambers.

### Example 8—Breast Tumor and Skin Model

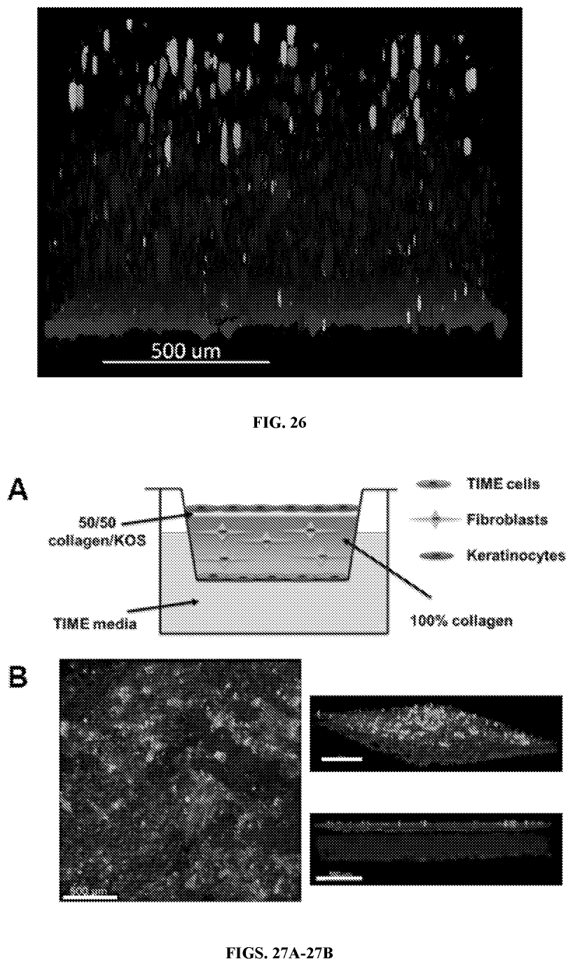

A vascularized multi-layer skin platform consisting of a dermal layer made of collagen seeded with normal human dermal fibroblasts (NHDFs) and an epidermal layer consisting of a collagen/keratin blend with immortalized keratinocytes was developed (FIG. 26). This multi-layer vascularized skin platform was combined with the breast tumor platform as a layer seeded underneath the skin platform. Keratinocyte differentiation was demostrated in a transwell system characteristic of a functioning epidermis as labeled by caspase 14, a differentiation marker for keratinocytes (FIG. 26). This system enables study of cancer cell invasion into the dermal layer of the skin and their interaction with healthy cells of skin including fibroblasts and keratinocytes.

An IBC in vitro tumor platform was created (FIG. 30) with functional surrounding blood and lymph vessels using subtractive tissue engineering methods. The platforms were fabricated by polymerizing a solution of 7 mg/ml collagen I seeded with cancer cells around two 22G needles. After polymerization and removal of the needles, a hollow void was left behind that was then filled with a solution of telomerase immortalized microvascular endothelial (TIME) cells and human dermal lymphatic endothelial cell (hDLEC) cells. Dual channel platforms were seeded with cancer cell lines derived from highly aggressive and metastatic breast cancer subtypes: IBC cell lines (SUM149, MDA-IBC3) and metastatic adenocarcinoma cell line MDA-MB-231 labeled with green fluorescent protein (GFP). Creation of an aligned functional blood and lymph vessel was achieved through a 72 hour flow protocol for the blood vessel and continuous perfusion of media at a rate of 0.01 dyne/cm2 will be used for the lymphatic vessel.

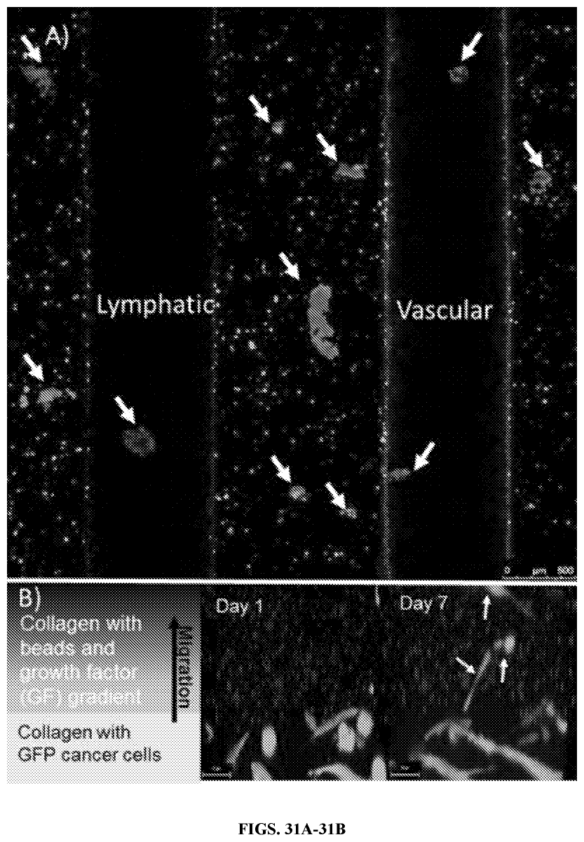

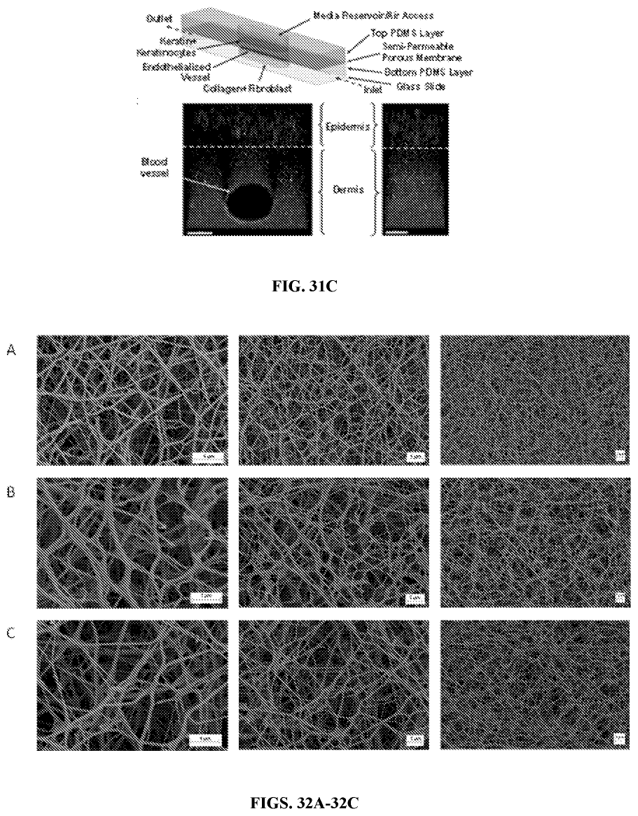

The permeability of blood and lymph vessels was tuned to promote cell migration into and out of these vessels. The influence of vessel leakiness on tumor invasion into the lymph and vascular channels was investigated. Migration of tumor cells, either as a collective migration of tumor emboli as clusters (IBC) or single cell migration (MDA-MB-231) into surrounding vessels is a well described aspect of metastases and treatment resistance in breast cancer with broader implications for other diseases. The single-channel tumor platform was used to study signaling between breast cancer cells within the collagen gel (MDA-MB-231, MDA-IBC3 and SUM 149) and a complete endothelium on the lumen (TIME cells) in response to fluid shear and demonstrated the role of cellular interactions on endothelial permeability as shown in FIG. 31. Types of breast tumor cells present and magnitude of wall shear stress ranging from 0.01-10 dynes/cm₂ were varied to cause cell-mediated and hemodynamic changes in permeability. Vessel permeability was measured by perfusing 70 kDa Oregon green-conjugated dextran into the channel and imaged to determine the average fluorescence intensity over time, which is proportional to the effective permeability coefficient. Endothelial permeability and integrity was analyzed by staining for the expression of CD31, VE-cadherin, LYVE-1, and Prox1. Immunofluorescent staining for E-cadherin expression was utilized to differentiate between collective cell migration compared to single cell migration (decreased E-cadherin expression). Cell invasiveness was quantified by determining rate of matrix degradation. DQ™-Collagen I solution (25 μg/mL) was mixed with collagen to a final concentration of 7 mg/ml and polymerized into channels. As the collagen channel is being degraded by cells, collagen fluoresced and proteolytic activity by tumor cells was determined by quantifying fluorescence intensity. Spatial and temporal cell migration was quantified through time lapse confocal imaging of fluorescently labeled tumor cells as shown in FIG. 32.

Combining the embodiments of the multi-layer skin platform and the single layer vascularized platforms as shown in FIG. 32, a multi-layer full thickness vascularized skin model was developed. The microfluidic platform comprises two polydimethylsiloxane (PDMS) layers separated by a PDMS porous membrane with 8 μm pore size (NiCo Form Inc.) as shown in FIG. 32A. PDMS layers were 1 mm thick and bonded following plasma treatment and assembled. The bottom layer had a cover slip to allow imaging and the top PDMS layer was exposed to the air-liquid interface to ensure differentiation of keratinocytes to develop the stratum corneum. The master mold of PDMS layers were fabricated using a micromilling method. PDMS layers included inlet and outlet access to enable filling of collagen seeded with fibroblasts at 0.1 million cells/ml as the bottom dermis and C/K gel with keratinocytes seeded on top of the porous membrane as the epidermis. Before polymerization, a needle was inserted through the dermal layer to create the microchannel and in which 0.2 million endothelial cells were injected twice through the channel. The vessel was preconditioned with increasing endothelial cell media flow rate for 3 days to create a confluent, functioning vessel (FIG. 32B).

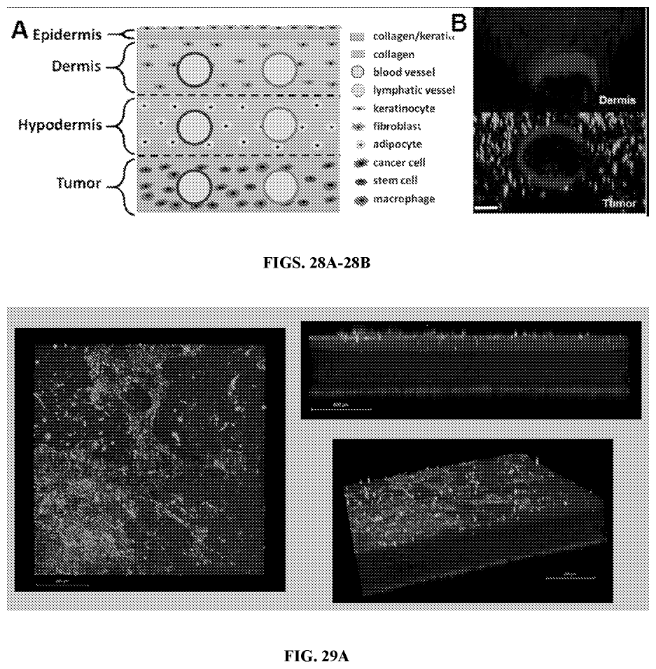

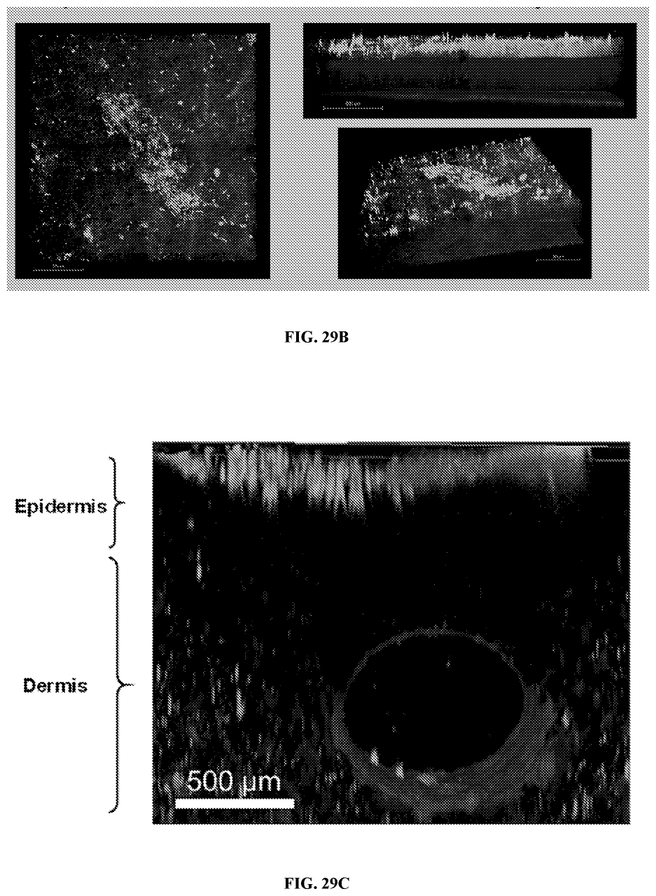

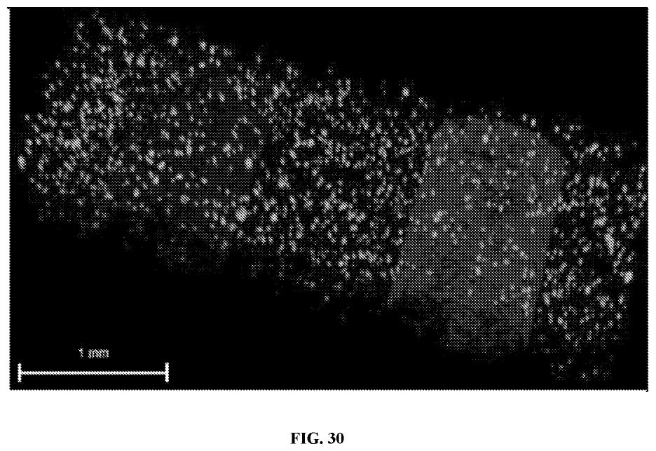

In order to create the comprehensive tumor platform, each layer was fabricated in succession from the bottom to the top. Each tissue layer was separated by a semipermeable membrane to promote integrity and distinct layers as shown in FIG. 28. Breast tissue is also composed of a significant amount of fat similar to subcutaneous fat in the skin called the hypodermis. A subcutaneous fat layer as added on top of the breast tumor and was composed of 1-2 mg/ml collagen seeded with adipocytes (0.1-0.5 million cells/ml). The vascularized skin platform containing both the epidermis and the dermis (FIG. 332C) was introduced on top of the hypodermis. To create the skin layer, NHDFs were seeded at densities ranging from 0.1-0.5 million cells/ml in lower density collagen with concentrations between 1.5 and 4 mg/ml. A semipermeable membrane coated with a matrix consisting of a 50/50 collagen/keratin (w %/w %) had keratinocytes seeded on top to form the epidermis. The tumor, hypodermis, and dermis each contained blood and lymph vessels undergoing the same preconditioning treatment as described in aim 1 to create a functional, aligned endothelium as shown by the schematic in FIG. 28. A vascularized breast skin platform as shown in FIG. 28B was created and expanded upon to include a hypodermis and epidermis to create the comprehensive tumor platform. The platform was cultured over 2 weeks to study tumor migration and invasion into the dermal layer.

### Example 9—Extracellular Matrix

Keratose Extraction: Keratose was extracted from human hair obtained from a local barber. Briefly, human hair was chopped into small pieces and soaked in a solution of 2 wt %/vol % paracetic acid (Sigma-Aldrich, St. Louis, Mo.) in DI water for 12 hours. Hair was then filtered from the liquid with a 500 μm sieve (W. S. Tyler, Mentor, Ohio) and rinsed to remove excess oxidant. Free proteins were extracted in excess 100 mM Tris base for 1 hour subsequently followed by DI water for 1 hour and transferred to a shaker at 37° C. at 180 rpm. Extracts were collected with the 500 μm sieve, neutralized, centrifuged, and finally filtered. Extracts were then dialyzed for 24 hours and lyophilized.

Cell Culture:

Primary normal human dermal fibroblasts (NHDFs, PromoCell, Heidelberg, Germany) were used in this study because of their ease of growth and handling and are also the most common and general cell type found in the healthy tissue. MDA-MB-231 breast cancer cells were also used to mimic cancerous tissue. Both cells were seeded in a t-75 Eppendorf HEPA-filtered flask (Eppendorf, Hamburg, Germany) and cultured in fibroblast basal medium 2 (PromoCell, Heidelberg, Germany) supplemented with 10 ml of fetal calf serum, 0.5 ml of human fibroblast growth factor, 2.5 ml of human insulin (PromoCell, Heidelberg, Germany), and 1% penicillin-streptomycin for NHDF and DMEM/F12 50:50 basal medium (Sigma Aldrich) supplemented with 10% fetal bovine serum (FBS) and 1% penicillin streptomycin for breast cancer cells. Cells were maintained in 5% CO₂ atmosphere at 37° C. in a sterile incubator. Media was changed every 2 days. Cells were detached for use in gels by aspirating media, neutralizing with 5 ml of phosphate buffered saline (PBS), and consequently aspirating the PBS. 3 ml of 0.04% trypsin (PromoCell, Heidelberg, Germany) for NHDF and 0.25% trypsin for breast cancer cells were added to the flask and incubated for 3 minutes. Trypsin/cell solution was neutralized with complete media and transferred to a 15 ml conical tube. Cells were centrifuged at 120 g for 5 minutes. Cell pellet was isolated and resuspended in 1 ml of complete media for cell-counting and use.

Collagen Gel Fabrication:

A working collagen solution of 1.5 and 3 mg/ml for NHDF, and 5 and 7 mg/ml for was prepared from the collagen stock solution by neutralizing with 10×DMEM (Sigma-Aldrich, St. Louis, Mo.), 1×DMEM (Gibco™ Gaithersburg, Md.) and 1 N NaOH (Fisher Scientific, Hampton, N.H.) that resulted in a pH of 7.4. For cell culture experiments, cells were embedded in the gel, resulting in a final concentration of 0.3 million and 0.5 million cells/ml for NHDF and breast cancer cells, respectively. The hydrogel solution was then added to cell culture-treated 96 well plates (Sigma-Aldrich, St. Louis, Mo.) and incubated at 37° C. for 40 minutes to allow polymerization. 100 μl of complete media was then added on top of each gel. Media was changed every 2 days.

Collagen/Keratose Gel Fabrication:

50/50 and 20/80 w %/w % collagen/KOS hydrogels were prepared by combining both stock and KOS dissolved in neutralizing buffer of equal volume. For example, to create a 3 mg/ml gel, 6 mg/ml (50/50) and 24 mg/ml (20/80) KOS in neutralizing buffer was prepared to mix with 6 mg/ml collagen stock. These different concentrations were employed for gels using varying stock collagen and C/K gel weight percentages (50/50 vs 20/80). Stock collagen and KOS/neutralilzing buffer solution was combined at 1:1 v:v ratio and mixed thoroughly with a spatula Gels were added to either cell culture-treated 96 well-plates and incubated at 37° C. for 40 minutes to allow polymerization. For cell culture experiments, gels were seeded with 6.5×10⁴ cells/ml and supplied with 100 μl of complete media. Media was changed every 2 days.

Fiber Diameter and Porosity Measured Using SEM Images:

SEM images of various acellular gels were taken to determine if there was any difference in porosity and fiber size. As observed qualitatively in FIG. 32, porosity seemed to increase with greater percentage of keratin and less collagen added to the sample. The fibers also started to bundle together as the collagen concentration decreased.

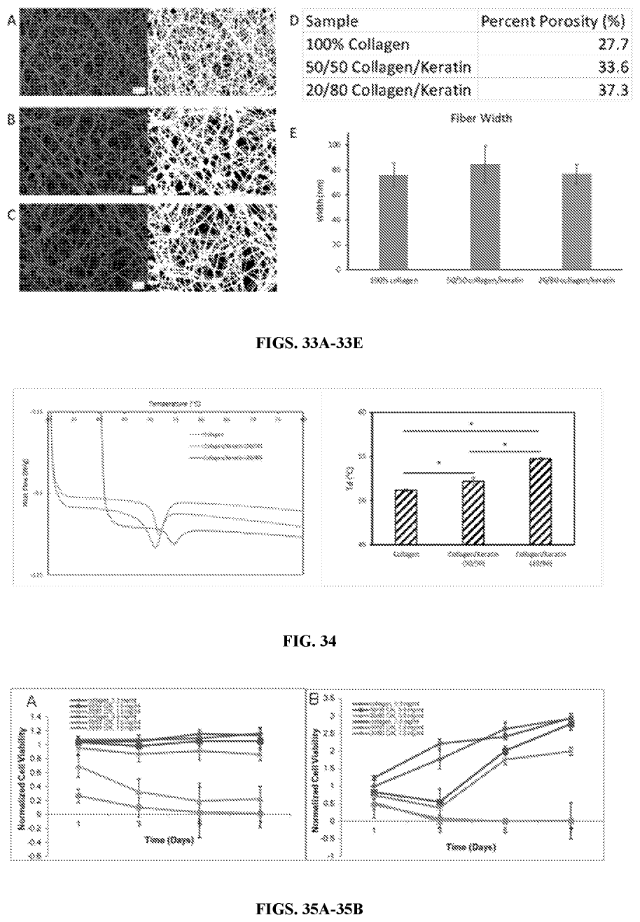

In order to determine quantitatively the porosity and fiber diameter of the various gels, ImageJ® was utilized. Porosity and fiber size is important in studying cell ECM interactions. Cells grown in a 3D gel can behave differently compared to a 2D plate due to the fiber network, allowing their morphology to change into a spindle shape. Collagen concentration can also influence material properties such as fiber structure, and in turn influencing cellular response. As shown in FIGS. 33A, B, and C, the same threshold was applied to all images to determine the percentage of black space (empty porous space) versus the white space (fibers). FIG. 33D shows the percent porosity of the gels, which validated that the increase in keratin concentration resulted in a higher percent porosity value. As keratin was added to the sample, less collagen was utilized, resulting in higher porosity. Fiber width was also measured with ImageJ® as shown in FIG. 33E. Fiber width ranged from 70 to 100 nm, but there was no significant difference observed between the averages of each gel. Average fiber width was calculated to be about 75 to 80 nm.

Thermally Stability Measurements by DSC and TGA:

DSC was utilized to determine whether addition of keratin increased the protein denaturation temperature when compared to 100% collagen. As shown in the FIG. 34A, single peaks occurred for each sample, demonstrating that the keratin and collagen polymerized homogenously when the gel was synthesized. The addition of keratin in the sample increased the protein denaturing temperature, with more keratin increasing the temperature even further in the 20/80 collagen/keratin gel. Significant difference with a p-value less than 0.01 was calculated between each sample (n=3). As discussed previously, increasing the protein denaturation temperature is critical in order to study the effect of heat on tissue and cell behavior. The results below demonstrate that gels that incorporate keratin can successfully be used in these studies.

Cell Viability of Fibroblast in Hydrogels:

Viability of fibroblasts in numerous gel types was measured using CellTiter Blue viability assay. As shown in FIG. 35A, viability in 50/50 collagen/KOS gels was comparable to that of 100% pure collagen gels. However, fibroblasts did not remain viable in 20/80 collagen/KOS hydrogels and started to die at day 3. At day 5, the viability of fibroblast decreased in both 100% collagen and 50/50 collagen/KOS gels and equilibriated by day 7. Similar results were demonstrated in 3 mg/ml hydrogels as shown in FIG. 35B.

Morphology and Growth of Fibroblasts in Hydrogels:

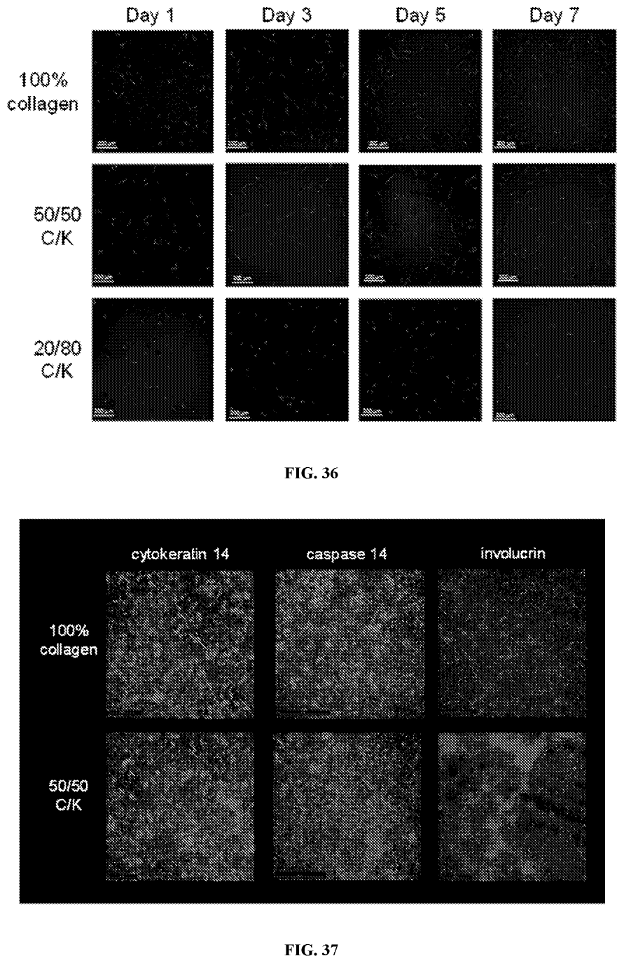

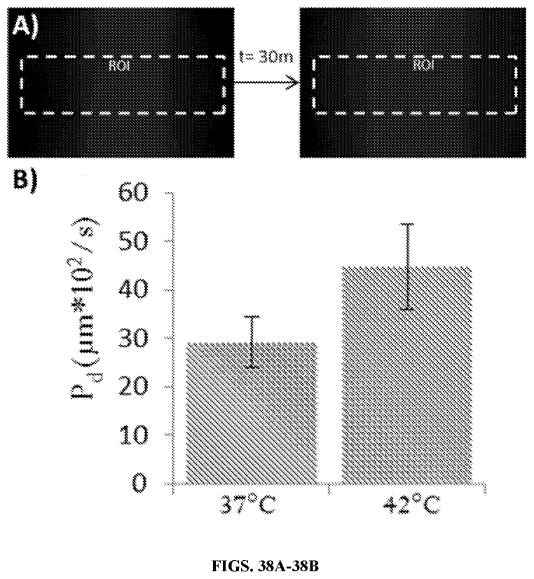

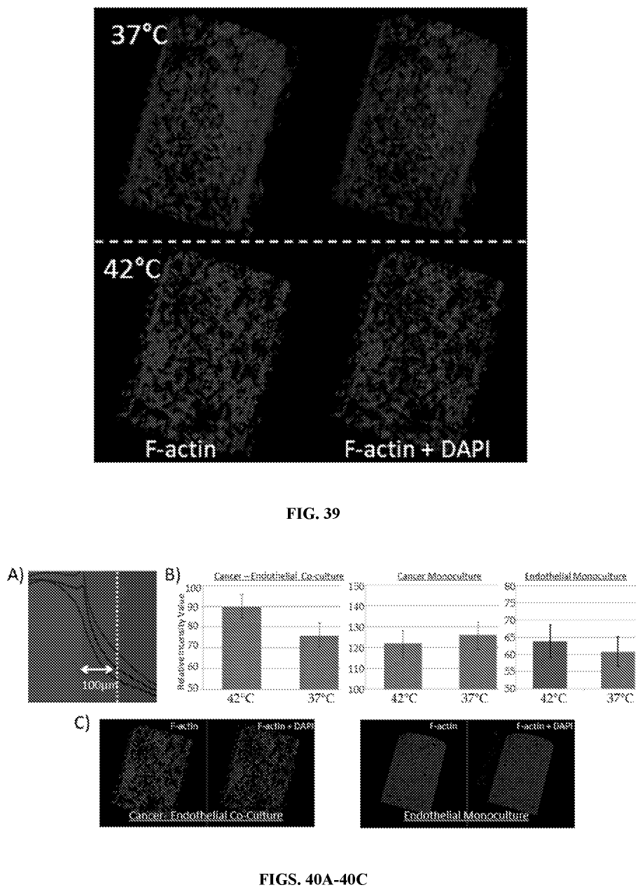

Morphology and growth were analyzed by confocal imaging of fibroblasts stained for actin over the course of 7 days. Fibroblasts proliferated and spread out over the course of 7 days in both the 100% collagen and 50/50 C/K gels. The morphology of the fibroblasts in the 50/50 C/K gel however is more spread out and elongated by day 7. Fibroblasts did not spread out and proliferate in the 20/80 C/K gel as shown in FIG. 36.

### Example 10—Tumor Hyperthermia

In an effort to overcome the complex barriers for nanoparticle delivery, recent preclinical and clinical studies have emphasized the significance of the tumor microenvironment as a potential therapeutic target to improve delivery into the tumor. These therapies often use focused physical perturbation of the tumor microenvironment to increase the targeting potential of systemic nanoparticles. More specifically, disrupting the tumor vasculature to enhance extravasation of systemic therapeutics into the tumor has been extensively investigated. While development effort has primarily focused on adjuvant therapeutics such as anti-angiogenesis antibodies or molecular approaches (VEGF, TNFα) to perturb the tumor vasculature and enhance extravasation of anti-neoplastic agents, physical or energy-based solutions (heat, acoustic energy, electroporation) are also currently explored. Despite the challenges associated with nanoparticle photothermal ablations, there remains great promise to utilize these particles for tumor-localized mild hyperthermia (41-45° C.) that enhances nanoparticle transport by modulating the tumor vasculature and dense extracellular matrix (ECM) of the tumor microenvironment. Mild hyperthermia has previously been shown to increase tumor blood flow and tumor microvascular pore size (<400 nm), which can amplify transvascular nanoparticle mass transport into the tumor where particles can accumulate owing to the dysfunctional lymphatic clearance. Additionally, local heating has been shown to strongly affect collagen fiber structure and mechanical properties which improves penetration through the tumor ECM.

In this study, the effects of mild hyperthermia (42° C.) were compared on the mass transport of SWNHs in a traditional 2D cell culture model and a set of relatively simple and high throughput 3D platforms. The results of this study highlight the potential mechanism of synergy between mild hyperthermia and nanoparticle transport and demonstrate the inability of 2D cell cultures, and oversimplified 3D cultures to probe these nanoparticle transport dynamics. After initially characterizing the SWNHs, it was demonstrated that SWNH transport in the tumor microenvironment is primarily enhanced by thermal targeting of an already leaky tumor vasculature, mirroring results in previous in vivo studies. Importantly, as a TE with an internal microfluidic channel was utilized with physiological fluid flows and barriers to nanoparticle drug-delivery (extravasation, diffusion into ECM, and cellular uptake), it was demonstrated that an increase in SWNH permeability exclusively results in higher concentrations into the tumor space in a tumor-vascular co-culture setup. As such, there is potential for further refinement for selective tumor-specific nanoparticle transport enhancement that limits drug delivery to off target tissue. Together, this study highlights the importance of early studies of nanoparticle transport in the tumor microenvironment when nanoparticles are utilized as co-delivery vehicles for generating hyperthermia and delivering drug payloads and demonstrates that these studies can be accomplished using relatively simple and inexpensive 3D TE models.

Increased vascular permeability that occurs as a result of a dynamic endothelial response to mild hyperthermia would enhance nanoparticle extravasation from the vasculature, this increase may not result insignificant enhancement of nanoparticle penetration deep into the tumor interstitial space. It has previously been shown that nanoparticles between 100-300 nm in diameter would be expected to be able to diffuse 100 μm into the tumor after extravasating from the tumor vasculature. As successful nanoparticle-based drug delivery therapies would require sufficient nanoparticle transport to this distance, the present study focused on understanding how mild hyperthermia enhances SWNH-QD penetration 100 μm into the platform.

FIG. 40A demonstrates the radial profile from a high resolution image taken during SWNH-QD perfusion into the co-culture platform, where a red signal (QDs) are primarily contained by the endothelial wall. Nanoparticles were perfused into the central vessel and a radial plot of intensity value from the center of the vessel was created. For direct comparison between samples the average relative SWNH-QD intensity values were calculated 100 μm from the endothelial monolayer. The relative signal intensity calculated for the tumor-endothelial co-culture platform are displayed in FIG. 40B (left), where average intensity values of 90.1±6.7 and 75±5.8 for 42° C. and 37° C. were determined respectively 100 μm from the vessel. Notably, the enhancement of vascular permeability during exposure to mild hyperthermia, resulted in a significant increase of SWNH-QD signal intensity deep into the tumor platform (p<0.05, Students T-test). FIG. 40B (middle) demonstrates that no significant changes in relative intensity values are seen in the tumor monoculture at the two temperatures (121.2±8.9 for 42° C. and 126.7±7.8 for 37° C.). Having demonstrated that the primary mechanism of thermal enhancement of SWNH-QD transport is likely a vascular thermos response which increased both the permeability and, as a consequence, the penetration of the nanoparticles into the tumor, it was then assessed if this response is magnified or moderated in tumors compared to healthy tissue. Therefore, an endothelialized microvascular platform was created with no cancer cells present in the matrix to represent a healthy vessel. The results from the endothelial monoculture are shown in FIG. 40B (right), where the average intensity value 100 μm from the vessel for platforms exposed at 42° C. is 64.1±5.1 and 60.7±4.4 for the 37° C. sample.

As no difference was observed between the two groups at different temperatures, it was sought to understand the difference in vascular response between the co-culture and endothelial monoculture microfluidic models by comparing the destabilization of the endothelial monolayer. Representative images after staining for F-actin in samples exposed to 42° C. for 1 h are seen in FIG. 40C, highlighting that a significant collapse of F-actin appearance in the endothelial layer is only observed in the co-culture platform. The endothelial monoculture platform exhibited perfectly intact and aligned cytoskeleton network throughout the vessel and staining with DAPI indicates that only cytoskeletal changes are present in the cancer-endothelial co-culture, as endothelial cell coverage is not significantly different between the co-culture and endothelial monoculture groups. The findings from these results indicates the possibility that hyperthermia affects nanoparticle transport only in the co-culture system, likely compounding vascular permeability changes inherent to the tumor microenvironment and highlights the promise for heat-enhanced nanoparticle transport that is localized to the tumor microenvironment.

All of the methods disclosed and claimed herein can be made and executed without undue experimentation in light of the present disclosure. While the compositions and methods of this invention have been described in terms of preferred embodiments, it will be apparent to those of skill in the art that variations may be applied to the methods and in the steps or in the sequence of steps of the method described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

### REFERENCES

The following references, to the extent that they provide exemplary procedural or other details supplementary to those set forth herein, are specifically incorporated herein by reference.
Antoine et al., PloS one, 10, e0122500, 2015.
Buchanan et al., Cell Adh. Migr., 8, 517-24, 2014.
Buchanan et al., Tissue Engineering Part C: Methods, 20, 64-75, 2013.
Chen et al., IEEE Transactions on ultrasonics, ferroelectrics, and frequency control, 43, 191-194, 1996.
Chrobak et al., Microvasc. Res., 71, 185-96, 2006.
Dong et al., PloS one, 10, e0115636, 2015.
Jain et al., Abeloff's Clinical Oncology (Fifth Edition), Elsevier, 108-126, 2014.
Landau and Lifshitz, Fluid Mechanics, Pergamon Books, Oxford, 1987.
Maria et al., J Diabetes Metab. S, 4, 003, 2011.
Pfeifer et al., PNAS, 1993.
Price et al., 2011.
Schedin and Keely, Cold Spring Harbor perspectives in biology, 3, a003228, 2011.
Siveen et al., British journal of cancer, 111, 1327, 2014.
Szot et al., Materials Science and Engineering: C, 31, 37-42, 2011.
Szot et al., Tissue Eng. Part C, 19, 864-74, 2013.
Tien, Curr. Opin. Chem. Eng., 3, 36-41, 2014.
Tong et al., Cancer Res, 2004.
Tourovskai et al., Exp. Biol. Med. (Maywood), 2014, 239, 1264-71, 2014.
Venkatasubramanian et al., Journal of theoretical biology, 253, 98-117, 2008.
Wang et al., Biomaterials, 27, 1924-1929, 2006.
Yeh et al., Ultrasound in Medicine and Biology, 28, 467-474, 2002.
Yuan et al., Cancer research, 55, 3752-3756, 1995.
Zervantonakis et al., Proceedings of the National Academy of Sciences, 109, 13515-13520, 2012.
Zheng et al., PNAS, 109, 9342-47, 2012.

What is claimed is:

1. A method of manufacturing a microfluidic device comprising:
(a) obtaining a base mold with at least one protruding chamber and at least one rod which spans from one edge of the mold through the chamber to the opposite edge of the mold;
(b) casting a polymer solution onto the base mold;
(c) curing the polymer solution to form a solidified polymer mold;
(d) bonding the solidified polymer mold to a surface;
(e) inserting extracellular matrix hydrogel into the chamber; and
(f) removing the at least one rod once the extracellular matrix hydrogel has polymerized, thereby producing a microfluidic device comprising at least one chamber with at least one channel running through said chamber, wherein the at least one channel comprises an inlet port and an outlet port.

2. The method of claim 1, wherein the base mold is an aluminum mold or polydimethylsiloxane (PDMS).

3. The method of claim 1 or claim 2, wherein obtaining the base mold comprises performing micro-milling using a computer-numerical-control (CNC) machining system.

4. The method of claim 1, wherein the chamber is cylindrical or rectangular.

5. The method of claim 1, wherein the rod is a needle.

6. The method of claim 5, wherein the needle is a 20-30 gauge needle.

7. The method of claim 1, wherein the at least one channel has a diameter of 100 to 1000 μm.

8. The method of claim 1, wherein the base mold comprises 2, 3, 4, or 5 chambers, wherein each chamber has at least one rod running through said chamber.

9. The method of claim 8, wherein the chambers are in parallel.

10. The method of claim 1 or claim 8, wherein at least one chamber has two rods running through said chamber.

11. The method of claim 10, wherein the two rods have different diameters.

12. The method of claim 11, wherein the two rods have the same diameter.

13. The method of claim 1, wherein the polymer solution comprises a silicon-based polymer.

14. The method of claim 2, wherein the silicon-based polymer is polydimethylsiloxane (PDMS).

15. The method of claim 1, wherein curing comprises applying heat to the polymer solution.

16. The method of claim 1, wherein bonding comprises plasma treatment.

17. The method of claim 1, wherein the surface is glass.

18. The method of claim 17, wherein the glass is further defined a glass coverslip.

19. The method of claim 1, further comprising treating the chamber with polyethleneimine (PEI) and/or glutaraldehyde before inserting the extracellular matrix hydrogel.

20. The method of claim 1, wherein the extracellular matrix hydrogel comprises elastin, keratin, fibrin, fibronectin, laminin, hyaluronic acid, and/or collagen.

21. The method of claim 1, wherein the extracellular matrix hydrogel comprises collagen.

22. The method of claim 15, wherein the collagen is type I collagen.

23. The method of claim 1, wherein the extracellular matrix hydrogel further comprises one or more populations of cells.

24. The method of claim 23, wherein the one or more populations of cells are selected from the group consisting of tumor cells, hepatocytes, cardiomyocytes, keratinocytes, fibroblasts, endothelial cells, stem cells, and macrophages.

25. The method of claim 1, further comprising injecting a population of cells into the channel.

26. The method of claim 25, wherein the population of cells comprises endothelial cells.

27. The method of claim 1, further comprising connecting the microfluidic device to a circulation system comprising one or more syringe pumps with controlled flow rates.

28. The method of claim 27, wherein two or more channels are connected to flow in parallel.

29. The method of claim 28, wherein the flow rate for each of the channels is distinct.

30. The method of claim 28, wherein the flow rate for each of the channels is essentially identical.

31. The method of claim 27, wherein two or more channels are connected to flow in series.

32. A microfluidic device comprising:
a polydimethylsiloxane (PDMS) scaffold;
a channel disposed within said PDMS scaffold; and
at least one chamber in fluid communication with the channel, wherein the chamber comprises an extracellular matrix hydrogel surrounding the channel.

33. The microfluidic device of claim 32, wherein the chamber is located in an interior region of said PDMS scaffold.

34. The microfluidic device of claim 33, wherein the channel extends from the chamber to an external surface of said PDMS scaffold.

35. The microfluidic device of claim 32, wherein the channel extends through said PDMS scaffold.

36. The microfluidic device of claim 32, wherein the device is produced according to claim 1.

37. The microfluidic device of claim 32, wherein the extracellular matrix hydrogel comprises elastin, fibrin, fibronectin, laminin, hyaluronic acid, keratin, and/or collagen.

38. The microfluidic device of claim 32, wherein the extracellular matrix hydrogel comprises collagen.

39. The microfluidic device of claim 38, wherein the collagen is type I collagen.

40. The microfluidic device of claim 38 or 39, wherein the collagen is present in the hydrogel at a concentration of 5 to 15 mg/mL.

41. The microfluidic device of claim 38 or 39, wherein the collagen is present in the extracellular matrix hydrogel at a concentration of 6 to 12 mg/mL.

42. The microfluidic device of any one of claims 32-39, further comprising a population of cells dispersed within the extracellular matrix hydrogel of the chamber.

43. The microfluidic device of claim 42, wherein the population of cells comprises tumor cells, cardiovascular cells, macrophages, kupfer cells, stellate cells, and/or hepatocytes.

44. The microfluidic device of claim 32, wherein the device comprise 2, 3, 4, or 5 chambers, wherein each chamber comprises a separate channel.

45. The microfluidic device of claim 44, wherein each chamber comprises a distinct population of cells within the extracellular matrix hydrogel.

46. The microfluidic device of claim 32, wherein the at least one chamber comprises two channels.

47. The microfluidic device of claim 46, wherein the two channels have distinct diameters.

48. The microfluidic device of claim 46, wherein the two channels have essentially identical diameters.

49. The microfluidic device of claim 32 or claim 46, wherein the diameter of the channel is between 100 to 500 μm.

50. The microfluidic device of claim 32, wherein the channel comprises a population of cells.

51. The microfluidic device of claim 50, wherein the population of cells comprises endothelial cells.

52. The microfluidic device of any one of claims 42-51, wherein the population of cells comprises at least 1,000 cells.

53. The microfluidic device of any one of claims 42-51, wherein the population of cells comprises at least 100,000 cells.

54. The microfluidic device of any one of claims 42-51, wherein the cells within population comprise detectable markers.

55. The microfluidic device of any one of claims 42-51, wherein the device comprises one population of cells within the extracellular matrix hydrogel of the chamber and a second population of cells within the channel.

56. The microfluidic device of any one of claims 42-51, wherein the device comprises one population of cells within the extracellular matrix hydrogel of the chamber and a second population of cells within the channel.

57. The microfluidic device of any one of claims 42-51, wherein the device comprises tumor cells within the extracellular matrix hydrogel of the chamber and endothelial cells within the channel.

58. The microfluidic device of claim 54, wherein the first population of cells comprise a detectable marker distinct from the marker of the second population of cells.

59. A method of evaluating a therapeutic or diagnostic agent comprising introducing the therapeutic agent or diagnostic agent to the flow of the microfluidic device of any one of claims 32-58 and characterizing the effect of said therapeutic agent or diagnostic agent.

60. The method of claim 59, wherein evaluating comprises monitoring transport, uptake, toxicity, and/or efficacy of the therapeutic agent.

61. The method of claim 59, wherein characterizing is further defined as measuring cell viability, cell morphology, cell proliferation, and/or enzyme secretion.

62. A method of measuring migration of a molecule comprising:
(a) obtaining a microfluidic device according to claim 32, wherein the device comprises a chamber with at least two channels running through said chamber and a region of extracellular matrix hydrogel comprising a population of cells between said at least two channels;
(b) introducing media to the flow of the device; and
(c) monitoring the migration of the molecule in the device.

63. The method of claim 62, wherein the molecule is a cell, particle, bacteria, chemical, nanoparticle, or toxicant.

64. The method of claim 62, wherein the channels comprise endothelial cells and the hydrogel comprises tumor cells and/or fibroblasts.

65. The method of claim 64, wherein the hydrogel further comprises macrophages.

66. The method of claim 62, wherein the hydrogel comprises keratinocytes, fibroblasts, adipocytes, endothelial cells, and/or tumor cells.

67. The method of claim 62, wherein the media comprises cells, growth factors, cytokines, hormones, antibodies, drugs, and/or enzymes.

68. A multi-layer hydrogel system for modeling skin comprising a first layer of collagen hydrogel comprising keratinocytes, a second layer of collagen hydrogel comprising fibroblasts and/or endothelial cells, and a third layer of collagen hydrogel comprising adipocytes, endothelial cells, an endothelial blood vessel, and/or lymph channels.

69. The system of claim 68, wherein the first, second, and/or third hydrogel layer further comprises keratin, melanocytes, hair follicles, and/or neural cells.

70. The system of claim 68, wherein the first, second, and/or third hydrogel layer comprises collagen and keratose.

71. The system of claim 68, further comprising a microfluidic device of any of claims 32-58.

72. The system of claim 71, wherein the microfluidic device is further defined as a tumor model.

73. The system of claim 72, wherein the tumor model is further defined as a breast cancer model.

74. A method of using the system of any of claims 68-73 for assessing the effect of a cell, drug, or external stimuli.
